SEQUENCE LISTING (46 sequences)

SEQ ID NO: 1 - DNA, 25 bp, synthetic construct
  source 1..25: mol_type = other DNA
  misc_feature order(12,13): note = DNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..13,14..24): mod_base = OTHER; note = Phosphorothioate linkage
  modified_base 13^14: mod_base = OTHER; note = Methylphosphonate linkage
  misc_feature 1..25: note = RM4000
  misc_feature order(1..11,14..25): note = RNA
  modified_base 11: mod_base = OTHER; note = 2-Prime-O-Methoxyethyl modified 5-methyl Cytidine
gaaagcaatgccatcacctcccctg

SEQ ID NO: 2 - DNA, 20 bp, synthetic construct
  source 1..20: mol_type = other DNA
agtcctctcccaagtccaca

SEQ ID NO: 3 - DNA, 20 bp, synthetic construct
  source 1..20: mol_type = other DNA
ggcacgaaggctcatcattc

SEQ ID NO: 4 - DNA, 21 bp, synthetic construct
  source 1..21: mol_type = other DNA
  modified_base 7..9: mod_base = OTHER; note = Locked Nucleic Acid
aggtgatggcattgctttcgt

SEQ ID NO: 5 - DNA, 23 bp, synthetic construct
  source 1..23: mol_type = other DNA
  modified_base order(3,7..9): mod_base = OTHER; note = Locked Nucleic Acid
aggtgatagcattgctttcgtgt

SEQ ID NO: 6 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base 1^2: mod_base = OTHER; note = Mesylphosphoramidate linkage
  modified_base order(2..13,14..24): mod_base = OTHER; note = Phosphorothioate linkage
  modified_base 13^14: mod_base = OTHER; note = Methylphosphonate linkage
  modified_base 24^25: mod_base = OTHER; note = (1,3-dimethylimidazolidin-2-ylidene) phosphoramidate linkage
  modified_base 11: mod_base = OTHER; note = 2-Prime-O-Methoxyethyl modified 5-methyl Cytidine
  misc_feature 1..25: note = RM4875
gaaagcaatgccatcacctcccctg

SEQ ID NO: 7 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base 2^3: mod_base = OTHER; note = Mesylphosphoramidate linkage
  modified_base order(3..13,14..24): mod_base = OTHER; note = Phosphorothioate linkage
  modified_base 13^14: mod_base = OTHER; note = Methylphosphonate linkage
  modified_base 11: mod_base = OTHER; note = 2-Prime-O-Methoxyethyl modified 5-methyl Cytidine
  misc_feature 1..25: note = RM4876
gaaagcaatgccatcacctcccctg

SEQ ID NO: 8 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2^3,4..13,14..24): mod_base = OTHER; note = Phosphorothioate linkage
  modified_base 3^4: mod_base = OTHER; note = Mesylphosphoramidate linkage
  modified_base 13^14: mod_base = OTHER; note = Methylphosphonate linkage
  modified_base 11: mod_base = OTHER; note = 2-Prime-O-Methoxyethyl modified 5-methyl Cytidine
  misc_feature 1..25: note = RM4877
gaaagcaatgccatcacctcccctg

SEQ ID NO: 9 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..4,5..13,14..24): mod_base = OTHER; note = Phosphorothioate linkage
  modified_base 4^5: mod_base = OTHER; note = Mesylphosphoramidate linkage
  modified_base 13^14: mod_base = OTHER; note = Methylphosphonate linkage
  modified_base 11: mod_base = OTHER; note = 2-Prime-O-Methoxyethyl modified 5-methyl Cytidine
  misc_feature 1..25: note = RM4878
gaaagcaatgccatcacctcccctg

SEQ ID NO: 10 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..5,6..13,14..24): mod_base = OTHER; note = Phosphorothioate linkage
  modified_base 5^6: mod_base = OTHER; note = Mesylphosphoramidate linkage
  modified_base 13^14: mod_base = OTHER; note = Methylphosphonate linkage
  modified_base 11: mod_base = OTHER; note = 2-Prime-O-Methoxyethyl modified 5-methyl Cytidine
  misc_feature 1..25: note = RM4879
gaaagcaatgccatcacctcccctg

SEQ ID NO: 11 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..6,7..13,14..24): mod_base = OTHER; note = Phosphorothioate linkage
  modified_base 6^7: mod_base = OTHER; note = Mesylphosphoramidate linkage
  modified_base 13^14: mod_base = OTHER; note = Methylphosphonate linkage
  modified_base 11: mod_base = OTHER; note = 2-Prime-O-Methoxyethyl modified 5-methyl Cytidine
  misc_feature 1..25: note = RM4880
gaaagcaatgccatcacctcccctg

SEQ ID NO: 12 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..7,8..13,14..24): mod_base = OTHER; note = Phosphorothioate linkage
  modified_base 7^8: mod_base = OTHER; note = Mesylphosphoramidate linkage
  modified_base 13^14: mod_base = OTHER; note = Methylphosphonate linkage
  modified_base 11: mod_base = OTHER; note = 2-Prime-O-Methoxyethyl modified 5-methyl Cytidine
  misc_feature 1..25: note = RM4881
gaaagcaatgccatcacctcccctg

SEQ ID NO: 13 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..8,9..13,14..24): mod_base = OTHER; note = Phosphorothioate linkage
  modified_base 8^9: mod_base = OTHER; note = Mesylphosphoramidate linkage
  modified_base 13^14: mod_base = OTHER; note = Methylphosphonate linkage
  modified_base 11: mod_base = OTHER; note = 2-Prime-O-Methoxyethyl modified 5-methyl Cytidine
  misc_feature 1..25: note = RM4882
gaaagcaatgccatcacctcccctg

SEQ ID NO: 14 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..9,10..13,14..24): mod_base = OTHER; note = Phosphorothioate linkage
  modified_base 9^10: mod_base = OTHER; note = Mesylphosphoramidate linkage
  modified_base 13^14: mod_base = OTHER; note = Methylphosphonate linkage
  modified_base 11: mod_base = OTHER; note = 2-Prime-O-Methoxyethyl modified 5-methyl Cytidine
  misc_feature 1..25: note = RM4883
gaaagcaatgccatcacctcccctg

SEQ ID NO: 15 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..10,11..13,14..24): mod_base = OTHER; note = Phosphorothioate linkage
  modified_base 10^11: mod_base = OTHER; note = Mesylphosphoramidate linkage
  modified_base 13^14: mod_base = OTHER; note = Methylphosphonate linkage
  modified_base 11: mod_base = OTHER; note = 2-Prime-O-Methoxyethyl modified 5-methyl Cytidine
  misc_feature 1..25: note = RM4884
gaaagcaatgccatcacctcccctg

SEQ ID NO: 16 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..11,12^13,14..24): mod_base = OTHER; note = Phosphorothioate linkage
  modified_base 11^12: mod_base = OTHER; note = Mesylphosphoramidate linkage
  modified_base 13^14: mod_base = OTHER; note = Methylphosphonate linkage
  modified_base 11: mod_base = OTHER; note = 2-Prime-O-Methoxyethyl modified 5-methyl Cytidine
  misc_feature 1..25: note = RM4885
gaaagcaatgccatcacctcccctg

SEQ ID NO: 17 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..12,14..24): mod_base = OTHER; note = Phosphorothioate linkage
  modified_base 12^13: mod_base = OTHER; note = Mesylphosphoramidate linkage
  modified_base 13^14: mod_base = OTHER; note = Methylphosphonate linkage
  modified_base 11: mod_base = OTHER; note = 2-Prime-O-Methoxyethyl modified 5-methyl Cytidine
  misc_feature 1..25: note = RM4886
gaaagcaatgccatcacctcccctg

SEQ ID NO: 18 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..13,14..24): mod_base = OTHER; note = Phosphorothioate linkage
  modified_base 13^14: mod_base = OTHER; note = Mesylphosphoramidate linkage
  modified_base 11: mod_base = OTHER; note = 2-Prime-O-Methoxyethyl modified 5-methyl Cytidine
  misc_feature 1..25: note = RM4887
gaaagcaatgccatcacctcccctg

SEQ ID NO: 19 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..13,15..24): mod_base = OTHER; note = Phosphorothioate linkage
  modified_base 13^14: mod_base = OTHER; note = Methylphosphonate linkage
  modified_base 14^15: mod_base = OTHER; note = Mesylphosphoramidate linkage
  modified_base 11: mod_base = OTHER; note = 2-Prime-O-Methoxyethyl modified 5-methyl Cytidine
  misc_feature 1..25: note = RM4888
gaaagcaatgccatcacctcccctg

SEQ ID NO: 20 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..13,14^15,16..24): mod_base = OTHER; note = Phosphorothioate linkage
  modified_base 13^14: mod_base = OTHER; note = Methylphosphonate linkage
  modified_base 15^16: mod_base = OTHER; note = Mesylphosphoramidate linkage
  modified_base 11: mod_base = OTHER; note = 2-Prime-O-Methoxyethyl modified 5-methyl Cytidine
  misc_feature 1..25: note = RM4889
gaaagcaatgccatcacctcccctg

SEQ ID NO: 21 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..13,14..16,17..24): mod_base = OTHER; note = Phosphorothioate linkage
  modified_base 13^14: mod_base = OTHER; note = Methylphosphonate linkage
  modified_base 16^17: mod_base = OTHER; note = Mesylphosphoramidate linkage
  modified_base 11: mod_base = OTHER; note = 2-Prime-O-Methoxyethyl modified 5-methyl Cytidine
  misc_feature 1..25: note = RM4890
gaaagcaatgccatcacctcccctg

SEQ ID NO: 22 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..13,14..17,18..24): mod_base = OTHER; note = Phosphorothioate linkage
  modified_base 13^14: mod_base = OTHER; note = Methylphosphonate linkage
  modified_base 17^18: mod_base = OTHER; note = Mesylphosphoramidate linkage
  modified_base 11: mod_base = OTHER; note = 2-Prime-O-Methoxyethyl modified 5-methyl Cytidine
  misc_feature 1..25: note = RM4891
gaaagcaatgccatcacctcccctg

SEQ ID NO: 23 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..13,14..18,19..24): mod_base = OTHER; note = Phosphorothioate linkage
  modified_base 13^14: mod_base = OTHER; note = Methylphosphonate linkage
  modified_base 18^19: mod_base = OTHER; note = Mesylphosphoramidate linkage
  modified_base 11: mod_base = OTHER; note = 2-Prime-O-Methoxyethyl modified 5-methyl Cytidine
  misc_feature 1..25: note = RM4892
gaaagcaatgccatcacctcccctg

SEQ ID NO: 24 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..13,14..19,20..24): mod_base = OTHER; note = Phosphorothioate linkage
  modified_base 13^14: mod_base = OTHER; note = Methylphosphonate linkage
  modified_base 19^20: mod_base = OTHER; note = Mesylphosphoramidate linkage
  modified_base 11: mod_base = OTHER; note = 2-Prime-O-Methoxyethyl modified 5-methyl Cytidine
  misc_feature 1..25: note = RM4893
gaaagcaatgccatcacctcccctg

SEQ ID NO: 25 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..13,14..20,21..24): mod_base = OTHER; note = Phosphorothioate linkage
  modified_base 13^14: mod_base = OTHER; note = Methylphosphonate linkage
  modified_base 20^21: mod_base = OTHER; note = Mesylphosphoramidate linkage
  modified_base 11: mod_base = OTHER; note = 2-Prime-O-Methoxyethyl modified 5-methyl Cytidine
  misc_feature 1..25: note = RM4894
gaaagcaatgccatcacctcccctg

SEQ ID NO: 26 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..13,14..21,22..24): mod_base = OTHER; note = Phosphorothioate linkage
  modified_base 13^14: mod_base = OTHER; note = Methylphosphonate linkage
  modified_base 21^22: mod_base = OTHER; note = Mesylphosphoramidate linkage
  modified_base 11: mod_base = OTHER; note = 2-Prime-O-Methoxyethyl modified 5-methyl Cytidine
  misc_feature 1..25: note = RM4895
gaaagcaatgccatcacctcccctg

SEQ ID NO: 27 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..13,14..22,23^24): mod_base = OTHER; note = Phosphorothioate linkage
  modified_base 13^14: mod_base = OTHER; note = Methylphosphonate linkage
  modified_base 22^23: mod_base = OTHER; note = Mesylphosphoramidate linkage
  modified_base 11: mod_base = OTHER; note = 2-Prime-O-Methoxyethyl modified 5-methyl Cytidine
  misc_feature 1..25: note = RM4896
gaaagcaatgccatcacctcccctg

SEQ ID NO: 28 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base order(1^2,24^25): mod_base = OTHER; note = (1,3-dimethylimidazolidin-2-ylidene) phosphoramidate linkage
  modified_base order(2..13,14..23): mod_base = OTHER; note = Phosphorothioate linkage
  modified_base 13^14: mod_base = OTHER; note = Methylphosphonate linkage
  modified_base 23^24: mod_base = OTHER; note = Mesylphosphoramidate linkage
  modified_base 11: mod_base = OTHER; note = 2-Prime-O-Methoxyethyl modified 5-methyl Cytidine
  misc_feature 1..25: note = RM4897
gaaagcaatgccatcacctcccctg

SEQ ID NO: 29 - DNA, 25 bp, synthetic construct
  source 1..25: mol_type = other DNA
  misc_feature order(12,13): note = DNA
  misc_feature order(1..11,14..25): note = RNA
  modified_base order(1..5,7,9,14,18,20,22..25): mod_base = OTHER; note = 2-Prime-O-Methyl modified nucleoside
  modified_base order(6,8,10,15,17,19,21): mod_base = OTHER; note = 2-Prime-Fluoro modified nucleoside
  modified_base 16: mod_base = OTHER; note = 2-Prime-O-Methoxyethyl modified nucleoside
  modified_base 1^2: mod_base = OTHER; note = (1,3-dimethylimidazolidin-2-ylidene) phosphoramidate linkage
  modified_base order(2..13,14..24): mod_base = OTHER; note = Phosphorothioate linkage
  modified_base 13^14: mod_base = OTHER; note = Methylphosphonate linkage
  modified_base 24^25: mod_base = OTHER; note = Mesylphosphoramidate linkage
  modified_base 11: mod_base = OTHER; note = 2-Prime-O-Methoxyethyl modified 5-methyl Cytidine
  misc_feature 1..25: note = RM4898
gaaagcaatgccatcacctcccctg

SEQ ID NO: 30 - DNA, 27 bp, synthetic construct
  source 1..27: mol_type = other DNA
  misc_feature order(14,15): note = DNA
  misc_feature order(1..13,16..27): note = RNA
  modified_base 2: mod_base = OTHER; note = 2-Prime-O-Methoxyethyl modified nucleoside
  modified_base order(4..6,8,10,12,16,19,21,23,25..27): mod_base = OTHER; note = 2-Prime-O-Methyl modified nucleoside
  modified_base order(7,9,11,17,20,22,24): mod_base = OTHER; note = 2-Prime-Fluoro modified nucleoside
  modified_base order(1^2,26^27): mod_base = OTHER; note = (1,3-dimethylimidazolidin-2-ylidene) phosphoramidate linkage
  modified_base order(4..13,14^15,17..26): mod_base = OTHER; note = Phosphorothioate linkage
  modified_base 15^16: mod_base = OTHER; note = Methylphosphonate linkage
  modified_base order(1,13,18): mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base 15: mod_base = OTHER; note = DNA nucleotide carrying a hypoxanthine nucleobase
  misc_feature 1..27: note = RM107361
catgatgccatacnntgccaccagggc

SEQ ID NO: 31 - DNA, 27 bp, synthetic construct
  source 1..27: mol_type = other DNA
  misc_feature order(13,14,15): note = DNA
  misc_feature order(1..12,16..27): note = RNA
  modified_base 2: mod_base = OTHER; note = 2-Prime-O-Methoxyethyl modified nucleoside
  modified_base order(4..6,8,10,12,16,19,21,23,25..27): mod_base = OTHER; note = 2-Prime-O-Methyl modified nucleoside
  modified_base order(7,9,11,17,20,22,24): mod_base = OTHER; note = 2-Prime-Fluoro modified nucleoside
  modified_base order(1^2,26^27): mod_base = OTHER; note = (1,3-dimethylimidazolidin-2-ylidene) phosphoramidate linkage
  modified_base order(4..15,17..26): mod_base = OTHER; note = Phosphorothioate linkage
  modified_base 15^16: mod_base = OTHER; note = Methylphosphonate linkage
  modified_base order(1,18): mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base 15: mod_base = OTHER; note = DNA nucleotide carrying a hypoxanthine nucleobase
  misc_feature 1..27: note = RM107362
catgatgccatacnntgccaccagggc

SEQ ID NO: 32 - DNA, 27 bp, synthetic construct
  source 1..27: mol_type = other DNA
  misc_feature order(13,14,15): note = DNA
  misc_feature order(1..12,16..27): note = RNA
  modified_base 2: mod_base = OTHER; note = 2-Prime-O-Methoxyethyl modified nucleoside
  modified_base order(4..6,8,10,12,16,19,21,23,25..27): mod_base = OTHER; note = 2-Prime-O-Methyl modified nucleoside
  modified_base order(7,9,11,17,20,22,24): mod_base = OTHER; note = 2-Prime-Fluoro modified nucleoside
  modified_base order(1^2,26^27): mod_base = OTHER; note = (1,3-dimethylimidazolidin-2-ylidene) phosphoramidate linkage
  modified_base order(4..15,17..26): mod_base = OTHER; note = Phosphorothioate linkage
  modified_base 15^16: mod_base = OTHER; note = Mesylphosphoramidate linkage
  modified_base order(1,18): mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base 15: mod_base = OTHER; note = DNA nucleotide carrying a hypoxanthine nucleobase
  misc_feature 1..27: note = RM107363
catgatgccatacnntgccaccagggc

SEQ ID NO: 33 - DNA, 27 bp, synthetic construct
  source 1..27: mol_type = other DNA
  misc_feature order(14,15): note = DNA
  misc_feature order(1..13,16..27): note = RNA
  modified_base 2: mod_base = OTHER; note = 2-Prime-O-Methoxyethyl modified nucleoside
  modified_base order(4..6,8,10,12,16,19,21,23,25..27): mod_base = OTHER; note = 2-Prime-O-Methyl modified nucleoside
  modified_base order(7,9,11,17,20,22,24): mod_base = OTHER; note = 2-Prime-Fluoro modified nucleoside
  modified_base order(1^2,15^16,26^27): mod_base = OTHER; note = (1,3-dimethylimidazolidin-2-ylidene) phosphoramidate linkage
  modified_base order(4..13,14^15,17..26): mod_base = OTHER; note = Phosphorothioate linkage
  modified_base order(1,13,18): mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base 15: mod_base = OTHER; note = DNA nucleotide carrying a hypoxanthine nucleobase
  misc_feature 1..27: note = RM107364
catgatgccatacnntgccaccagggc

SEQ ID NO: 34 - DNA, 27 bp, synthetic construct
  source 1..27: mol_type = other DNA
  misc_feature order(14,15): note = DNA
  misc_feature order(1..13,16..27): note = RNA
  modified_base 2: mod_base = OTHER; note = 2-Prime-O-Methoxyethyl modified nucleoside
  modified_base order(4..6,8,10,12,16,19,21,23,25..27): mod_base = OTHER; note = 2-Prime-O-Methyl modified nucleoside
  modified_base order(7,9,11,17,20,22,24): mod_base = OTHER; note = 2-Prime-Fluoro modified nucleoside
  modified_base order(1^2,26^27): mod_base = OTHER; note = (1,3-dimethylimidazolidin-2-ylidene) phosphoramidate linkage
  modified_base order(4..13,14^15,17..26): mod_base = OTHER; note = Phosphorothioate linkage
  modified_base 15^16: mod_base = OTHER; note = Mesylphosphoramidate linkage
  modified_base order(1,13,18): mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base 15: mod_base = OTHER; note = DNA nucleotide carrying a hypoxanthine nucleobase
  misc_feature 1..27: note = RM107365
catgatgccatacnntgccaccagggc

SEQ ID NO: 35 - DNA, 27 bp, synthetic construct
  source 1..27: mol_type = other DNA
  misc_feature order(14,15): note = DNA
  misc_feature order(1..13,16..27): note = RNA
  modified_base 2: mod_base = OTHER; note = 2-Prime-O-Methoxyethyl modified nucleoside
  modified_base order(4..6,8,10,12,16,19,21,23,25..27): mod_base = OTHER; note = 2-Prime-O-Methyl modified nucleoside
  modified_base order(7,9,11,17,20,22,24): mod_base = OTHER; note = 2-Prime-Fluoro modified nucleoside
  modified_base order(1^2,26^27): mod_base = OTHER; note = Mesylphosphoramidate linkage
  modified_base order(4..13,14^15,17..26): mod_base = OTHER; note = Phosphorothioate linkage
  modified_base 15^16: mod_base = OTHER; note = Methylphosphonate linkage
  modified_base order(1,13,18): mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base 15: mod_base = OTHER; note = DNA nucleotide carrying a hypoxanthine nucleobase
  misc_feature 1..27: note = RM107376
catgatgccatacnntgccaccagggc

SEQ ID NO: 36 - DNA, 27 bp, synthetic construct
  source 1..27: mol_type = other DNA
  misc_feature order(13,14,15): note = DNA
  misc_feature order(1..12,16..27): note = RNA
  modified_base 2: mod_base = OTHER; note = 2-Prime-O-Methoxyethyl modified nucleoside
  modified_base order(4..6,8,10,12,16,19,21,23,25..27): mod_base = OTHER; note = 2-Prime-O-Methyl modified nucleoside
  modified_base order(7,9,11,17,20,22,24): mod_base = OTHER; note = 2-Prime-Fluoro modified nucleoside
  modified_base order(1^2,26^27): mod_base = OTHER; note = Mesylphosphoramidate linkage
  modified_base order(4..15,17..26): mod_base = OTHER; note = Phosphorothioate linkage
  modified_base 15^16: mod_base = OTHER; note = Methylphosphonate linkage
  modified_base order(1,18): mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base 15: mod_base = OTHER; note = DNA nucleotide carrying a hypoxanthine nucleobase
  misc_feature 1..27: note = RM107377
catgatgccatacnntgccaccagggc

SEQ ID NO: 37 - DNA, 27 bp, synthetic construct
  source 1..27: mol_type = other DNA
  misc_feature order(13,14,15): note = DNA
  misc_feature order(1..12,16..27): note = RNA
  modified_base 2: mod_base = OTHER; note = 2-Prime-O-Methoxyethyl modified nucleoside
  modified_base order(4..6,8,10,12,16,19,21,23,25..27): mod_base = OTHER; note = 2-Prime-O-Methyl modified nucleoside
  modified_base order(7,9,11,17,20,22,24): mod_base = OTHER; note = 2-Prime-Fluoro modified nucleosidev
  modified_base order(1^2,15^16,26^27): mod_base = OTHER; note = Mesylphosphoramidate linkage
  modified_base order(4..15,17..26): mod_base = OTHER; note = Phosphorothioate linkage
  modified_base order(1,18): mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base 15: mod_base = OTHER; note = DNA nucleotide carrying a hypoxanthine nucleobase
  misc_feature 1..27: note = RM107378
catgatgccatacnntgccaccagggc

SEQ ID NO: 38 - DNA, 27 bp, synthetic construct
  source 1..27: mol_type = other DNA
  misc_feature order(14,15): note = DNA
  misc_feature order(1..13,16..27): note = RNA
  modified_base 2: mod_base = OTHER; note = 2-Prime-O-Methoxyethyl modified nucleoside
  modified_base order(4..6,8,10,12,16,19,21,23,25..27): mod_base = OTHER; note = 2-Prime-O-Methyl modified nucleoside
  modified_base order(7,9,11,17,20,22,24): mod_base = OTHER; note = 2-Prime-Fluoro modified nucleoside
  modified_base order(1^2,26^27): mod_base = OTHER; note = Mesylphosphoramidate linkage
  modified_base order(4..13,14^15,17..26): mod_base = OTHER; note = Phosphorothioate linkage
  modified_base 15^16: mod_base = OTHER; note = (1,3-dimethylimidazolidin-2-ylidene) phosphoramidate linkage
  modified_base order(1,13,18): mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base 15: mod_base = OTHER; note = DNA nucleotide carrying a hypoxanthine nucleobase
  misc_feature 1..27: note = RM107379
catgatgccatacnntgccaccagggc

SEQ ID NO: 39 - DNA, 27 bp, synthetic construct
  source 1..27: mol_type = other DNA
  misc_feature order(14,15): note = DNA
  misc_feature order(1..13,16..27): note = RNA
  modified_base 2: mod_base = OTHER; note = 2-Prime-O-Methoxyethyl modified nucleoside
  modified_base order(4..6,8,10,12,16,19,21,23,25..27): mod_base = OTHER; note = 2-Prime-O-Methyl modified nucleoside
  modified_base order(7,9,11,17,20,22,24): mod_base = OTHER; note = 2-Prime-Fluoro modified nucleoside
  modified_base order(1^2,15^16,26^27): mod_base = OTHER; note = Mesylphosphoramidate linkage
  modified_base order(4..13,14^15,17..26): mod_base = OTHER; note = Phosphorothioate linkage
  modified_base order(1,13,18): mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base 15: mod_base = OTHER; note = DNA nucleotide carrying a hypoxanthine nucleobase
  misc_feature 1..27: note = RM107380
catgatgccatacnntgccaccagggc

SEQ ID NO: 40 - DNA, 25 bp, synthetic construct
  source 1..25: mol_type = other DNA
  misc_feature order(14,15): note = DNA
  misc_feature order(1..13,16..25): note = RNA
  modified_base 2: mod_base = OTHER; note = 2-Prime-O-Methoxyethyl modified nucleoside
  modified_base order(4..6,8,10,12,16,19,21,23,25): mod_base = OTHER; note = 2-Prime-O-Methyl modified nucleoside
  modified_base order(7,9,11,17,20,22,24): mod_base = OTHER; note = 2-Prime-Fluoro modified nucleoside
  modified_base order(1^2,24^25): mod_base = OTHER; note = (1,3-dimethylimidazolidin-2-ylidene) phosphoramidate linkage
  modified_base order(4..13,14^15,17..24): mod_base = OTHER; note = Phosphorothioate linkage
  modified_base 15^16: mod_base = OTHER; note = Methylphosphonate linkage
  modified_base order(1,13,18): mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base 15: mod_base = OTHER; note = DNA nucleotide carrying a hypoxanthine nucleobase
  misc_feature 1..25: note = RM107382
catgatgccatacnntgccaccagg

SEQ ID NO: 41 - DNA, 25 bp, synthetic construct
  source 1..25: mol_type = other DNA
  misc_feature order(14,15): note = DNA
  misc_feature order(1..13,16..25): note = RNA
  modified_base 2: mod_base = OTHER; note = 2-Prime-O-Methoxyethyl modified nucleoside
  modified_base order(4..6,8,10,12,16,19,21,23,25): mod_base = OTHER; note = 2-Prime-O-Methyl modified nucleoside
  modified_base order(7,9,11,17,20,22,24): mod_base = OTHER; note = 2-Prime-Fluoro modified nucleoside
  modified_base order(1^2,24^25): mod_base = OTHER; note = Mesylphosphoramidate linkage
  modified_base order(4..13,14^15,17..24): mod_base = OTHER; note = Phosphorothioate linkage
  modified_base 15^16: mod_base = OTHER; note = Methylphosphonate linkage
  modified_base order(1,13,18): mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base 15: mod_base = OTHER; note = DNA nucleotide carrying a hypoxanthine nucleobase
  misc_feature 1..25: note = RM107385
catgatgccatacnntgccaccagg

SEQ ID NO: 42 - DNA, 22 bp, synthetic construct
  source 1..22: mol_type = other DNA
gttcagcaagatcaaggctcac

SEQ ID NO: 43 - DNA, 23 bp, synthetic construct
  source 1..23: mol_type = other DNA
  modified_base order(5,10..12): mod_base = OTHER; note = Locked Nucleic Acid
ctggtggcacggtatggcatcat

SEQ ID NO: 44 - DNA, 23 bp, synthetic construct
  source 1..23: mol_type = other DNA
  modified_base order(5,10..12,20): mod_base = OTHER; note = Locked Nucleic Acid
ctggtggcacagtatggcatcat

SEQ ID NO: 45 - DNA, 23 bp, synthetic construct
  source 1..23: mol_type = other DNA
  modified_base order(5,10..12,20): mod_base = OTHER; note = Locked Nucleic Acid
ctggtggcacagtatggcatcat

SEQ ID NO: 46 - DNA, 21 bp, synthetic construct
  source 1..21: mol_type = other DNA
tggagcaggtggtcatcacaa

(19) United States
(12) Patent Application Publication
(10) Pub. No.: US 2026/0275338 A1
(43) Pub. Date: Sep. 17, 2026

(54) CHEMICALLY MODIFIED ANTISENSE OLIGONUCLEOTIDES FOR USE IN RNA EDITING

(71) Applicant: ProQR Therapeutics II B.V., Leiden (NL)

(72) Inventors: Háfrun HÁLFDÁNARDÓTTIR, Leiden (NL); Peggy Ramona BOHLÄNDER, Leiden (NL); Peter Christian DE VISSER, Leiden (NL); Marko POTMAN, Leiden (NL); Sjoerd Johannes RIEKEN, Leiden (NL)

(73) Assignee: ProQR Therapeutics II B.V., Leiden (NL)

(21) Appl. No.: 19/168,346

(22) PCT Filed: Mar. 22, 2024
(86) PCT No.: PCT/EP2024/057800

§ 371 Date: Sep. 24, 2025

(30) Foreign Application Priority Data
Mar. 24, 2023 (GB) 2304363.1

(51) Int. Cl. C12N 15/113

(52) U.S. Cl. CPC C12N 15/113; C12N 2310/11; C12N 2310/314; C12N 2310/315; C12N 2310/321; C12N 2310/322; C12N 2310/333; C12N 2310/334; C12N 2310/346

(57) ABSTRACT

The invention relates to the field of RNA editing using antisense oligonucleotides (AONs) that comprise at least one non-naturally occurring internucleoside linkage modification. The RNA editing is directed at deaminating target adenosines in endogenously present RNA nucleic acid molecules, such as pre-mRNA and mRNA transcript products, using deaminating enzymes such as ADAR1 and ADAR2 that are preferably endogenously present in the cell. The non-natural internucleoside linkage modification is a phosphoramidate linkage, preferably a mesyl phosphoramidate (PNms) linkage.

### TECHNICAL FIELD

The invention relates to the field of medicine, and in particular to the field of RNA editing, whereby an RNA molecule in a cell is targeted by an antisense oligonucleotide (AON) to specifically change a target nucleotide present in the target RNA molecule. The invention is aimed at amending a specific nucleotide, such as a mutated nucleotide that may cause disease, in the target RNA molecule by engaging an enzyme having deaminase activity. Disclosed herein are AONs that are chemically modified to increase their in vivo and in vitro stability, and thereby increase the efficiency and level of RNA editing.

### BACKGROUND

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called Adenosine Deaminases acting on RNA (ADAR) and APOBEC/AID (cytidine deaminases that act on RNA), respectively.

ADAR is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an A into I in a nearby, predefined, position in the target RNA, by deamination of the nucleobase. Inosine is read as guanosine by the translational machinery of the cell, meaning that, if an edited adenosine is in a coding region of an mRNA or pre-mRNA, it can recode the protein sequence. A-to-I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result, exons may be included or skipped. The enzymes catalysing adenosine deamination are within an enzyme family of ADARs, which include human deaminases hADAR1 and hADAR2, as well as hADAR3. However, for hADAR3 no deaminase activity has been demonstrated.

The use of oligonucleotides to edit a target RNA applying adenosine deaminase has been described (e.g., Woolf et al. 1995. Proc Natl Acad Sci USA 92:8298-8302; Montiel-Gonzalez et al. 2013. Proc Natl Acad Sci USA 110(45):18285-18290; Vogel et al. 2014. Angewandte Chemie Int Ed 53:267-271). A disadvantage of the method described by Montiel-Gonzalez et al. (2013) is the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014) suffers from similar drawbacks, in that it is not clear how to apply the system without having to genetically modify the ADAR first and subsequently transfect or transform the cells harboring the target RNA, to provide the cells with this genetically engineered protein. U.S. Pat. No. 9,650,627 describes a similar system. The oligonucleotides of Woolf et al. (1995) that were 100% complementary to the target RNA sequences suffered from severe lack of specificity: nearly all adenosines in the target RNA strand that was complementary to the antisense oligonucleotide were edited.

It is known that ADAR may act on any dsRNA. Through a process sometimes referred to as ‘promiscuous editing’, the enzyme will edit multiple A's in the dsRNA. Hence, there was a need for methods and means that circumvent such promiscuous editing and only target specific adenosines in a target RNA molecule to become therapeutic applicable. Vogel et al. (2014) showed that such off-target editing can be suppressed by using 2′-O-methyl (2′-OMe) modified nucleosides in the oligonucleotide at positions opposite to adenosines that should not be edited and used a non-modified nucleoside directly opposite to the specifically targeted adenosine on the target RNA. However, the specific editing effect at the target nucleotide has not been shown to take place without the use of recombinant ADAR enzymes having covalent bonds with the AON. Several publications have now shown that the recruitment of endogenous ADAR (hence without the need for an exogenous and/or recombinant source) is feasible while maintaining a specificity in which a single adenosine within a target RNA molecule can be targeted and deaminated to an inosine. WO2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop/hairpin structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell (endogenously present) to the dsRNA formed by hybridization of the target sequence with the targeting portion. Due to the recruitment portion, there is no need for conjugated entities or presence of modified recombinant ADAR enzymes. WO2016/097212 describes the recruitment portion as being a stem-loop structure mimicking either a natural substrate (e.g., the GluB receptor) or a Z-DNA structure known to be recognized by the dsRNA binding domains, or Z-DNA binding domains, of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described is an intramolecular stem-loop structure, formed within the AON itself, and are thought to attract (endogenous) ADAR. Similar stem-loop structure-comprising systems for RNA editing have since then been described in WO2017/050306, WO2020/001793, WO2017/010556, U.S. Pat. No. 11,390,865, WO2020/246560, and WO2022/078995.

WO2017/220751 and WO2018/041973 describe a next generation type of AONs that do not comprise such a stem-loop structure but that are (almost fully) complementary to the targeted area, and that appeared still capable of attracting endogenous ADAR enzymes. In one embodiment, one or more mismatching nucleotides, wobbles, or bulges exist between the oligonucleotide and the target sequence. A sole mismatch may be at the site of the nucleoside opposite the target adenosine, but in other embodiments AONs (or “RNA editing oligonucleotides”—even though the deamination reaction is carried out by the ADAR enzyme—often abbreviated to ‘EONs’) were described with multiple bulges and/or wobbles when attached to the target sequence area. It appeared possible to achieve in vitro, ex vivo and in vivo RNA editing with AONs lacking a stem-loop structure and with endogenous ADAR enzymes when the sequence of the AON was carefully selected such that it could attract/recruit ADAR. The ‘orphan nucleoside’, which is defined as the nucleoside in the AON that is positioned directly opposite the target adenosine in the target RNA molecule, was a nucleotide with an unmodified cytosine nucleobase and that did not carry a 2′-OMe modification. The orphan nucleoside can be a deoxyribonucleoside (DNA), wherein the remainder of the AON could still carry 2′-O-alkyl modifications at the sugar entity (such as 2′-OMe), or the nucleotides directly surrounding the orphan nucleoside contained chemical modifications (such as DNA in comparison to RNA) that further improved the RNA editing efficiency and/or increased the resistance against nucleases. Such effects could even be further improved by using sense oligonucleotides (SONs) that ‘protected’ the AONs against breakdown upon delivery to the cells (described in WO2018/134301 and U.S. Pat. No. 11,274,300).

The use of chemical modifications and particular structures in oligonucleotides that could be used in ADAR-mediated editing of specific adenosines in a target RNA have been the subject of numerous disclosures in the field, such as WO2019/111957, WO2019/158475, WO2020/165077, WO2020/201406, WO2020/211780, WO2021/008447, WO2021/020550, WO2021/060527, WO2021/117729, WO2021/136408, WO2021/182474, WO2021/216853, WO2021/242778, WO2021/242870, WO2021/242889, WO2022/007803, WO2022/018207, WO2022/026928, and WO2022/124345. The use of specific sugar moieties has been disclosed in for instance WO2020/154342, WO2020/154343, WO2020/154344, WO2022/103839, and WO2022/103852, whereas the use of stereo-defined linker moieties (in general for oligonucleotides that for instance can be used for exon skipping, in gapmers, in siRNA, or specifically for RNA-editing oligonucleotides, related to a wide variety of target sequences) has been described in WO2011/005761, WO2014/010250, WO2014/012081, WO2015/107425, WO2017/015575 (HTT), WO2017/062862, WO2017/160741, WO2017/192664, WO2017/192679 (DMD), WO2017/198775, WO2017/210647, WO2018/067973, WO2018/098264, WO2018/223056 (PNPLA3), WO2018/223073 (APOC3), WO2018/223081 (PNPLA3), WO2018/237194, WO2019/032607 (C9orf72), WO2019/055951, WO2019/075357 (SMA/ALS), WO2019/200185 (DM1), WO2019/217784 (DM1), WO2019/219581, WO2020/118246 (DM1), WO2020/160336 (HTT), WO2020/191252, WO2020/196662, WO2020/219981 (USH2A), WO2020/219983 (RHO), WO2020/227691 (C9orf72), WO2021/071788 (C9orf72), WO2021/071858, WO2021/178237 (MAPT), WO2021/234459, WO2021/237223, WO2022/099159, WO2021/030778, WO2022/174053, and WO2023/278589. Next to these disclosures, an extensive number of publications relate to the targeting of specific RNA target molecules, or specific adenosines within such RNA target molecules, be it to repair a mutation that resulted in a premature stop codon, or other mutation causing disease. Examples of such disclosures in which adenosines are targeted within specified target RNA molecules are WO2020/157008 and WO2021/136404 (USH2A); WO2021/113270 (APP); WO2021/113390 (CMT1A); WO2021/209010 (IDUA, Hurler syndrome); WO2021/231673 and WO2021/242903 (LRRK2); WO2021/231675 (ASS1); WO2021/231679 (GJB2); WO2019/071274 and WO2021/231680 (MECP2); WO2021/231685 and WO2021/231692 (OTOF, autosomal recessive non-syndromic hearing loss); WO2021/231691 (XLRS); WO2021/231698 (argininosuccinate lyase deficiency); WO2021/130313 and WO2021/231830 (ABCA4); and WO2021/243023 (SERPINA1).

Despite the numerous efforts in the art to provide improved version of AONs that have a certain level of stability and that retain an ability to cause RNA editing after interacting with the target RNA molecule, there remains a need for further improved chemical modifications in such oligonucleotides that are conducive to RNA editing, which are less toxic (in general or specifically on the chemistry), provide more stability and/or provide more efficient RNA editing when administered to a cell and capable of recruiting endogenous deamination enzymes.

### SUMMARY OF THE INVENTION

Disclosed herein is an antisense oligonucleotide (AON) that is able to mediate adenosine deamination by recruitment of a deaminating enzyme in a cell after the AON has formed a double-stranded complex with a region of a target RNA nucleic acid molecule in a cell, wherein the region comprises a target adenosine, wherein the deaminating enzyme can deaminate the target adenosine into an inosine, and wherein the AON comprises one or more linkage modifications with a structure according to formula (I):

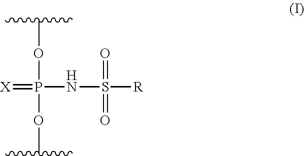

wherein:
X=O or S; and
R=an aryl, a substituted aryl, a heterocycle, a substituted heterocycle, an aromatic heterocycle, a substituted aromatic heterocycle, a C₁-C₆alkoxy, a substituted C₁-C₆ alkoxy, a C₁-C₂₀ alkyl, a substituted C₁-C₂₀ alkyl, a C₁-C₆ alkenyl, a C₁-C₆ substituted alkenyl, a C₁-C₆ alkynyl, a substituted C₁-C₆ alkynyl, or a conjugate group. In a preferred embodiment, X=O and R=methyl.

Disclosed herein is also an AON that is able to mediate adenosine deamination by recruitment of a deaminating enzyme in a cell after the AON has formed a double-stranded complex with a region of a target RNA nucleic acid molecule in a cell, wherein the region comprises a target adenosine, wherein the deaminating enzyme can deaminate the target adenosine into an inosine, and wherein the AON comprises a moiety at one and/or both termini with a structure according to formula (II):

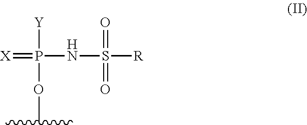

wherein: X=O or S;
Y=O⁻ or S⁻; and
R=an aryl, a substituted aryl, a heterocycle, a substituted heterocycle, an aromatic heterocycle, a substituted aromatic heterocycle, a C₁-C₆ alkoxy, a substituted C₁-C₆ alkoxy, a C₁-C₂₀ alkyl, a substituted C₁-C₂₀ alkyl, a C₁-C₆ alkenyl, a C₁-C₆ substituted alkenyl, a C₁-C₆ alkynyl, a substituted C₁-C₆ alkynyl, or a conjugate group. In a preferred embodiment, X=0 and R=methyl.

Disclosed herein is an AON comprising a sequence configured for the deamination of a target nucleotide, preferably an adenosine, in the target nucleic acid molecule, wherein the AON comprises at least one linkage according to the structure of formula (I) as described herein. In one aspect, the AON can engage an enzyme with deamination activity such as ADAR1 or ADAR2, and preferably the target nucleotide is an adenosine that is deaminated by the deaminating enzyme to an inosine.

Disclosed herein is an AON, wherein the nucleotide that is directly opposite the target adenosine is the orphan nucleotide, wherein the internucleoside linkage numbering is such that linkage number 0 is the linkage 5′ from the orphan nucleotide, the linkage positions in the oligonucleotide are positively (+) incremented towards the 5′ end and negatively (−) incremented towards the 3′ end, and wherein the linkage modification according to formula (I) is present at: linkage position −2; at the linkage position connecting the 5′ terminal two nucleosides in the AON; and/or at the linkage position connecting the 3′ terminal nucleosides in the AON.

Disclosed is also a pharmaceutical composition comprising an AON as disclosed herein, and a pharmaceutically acceptable carrier.

Disclosed is also an AON or a pharmaceutical composition for use in the treatment of a disorder, preferably a disorder of the liver, the Central Nervous System, a kidney disorder, or a cardiovascular disease. The disorder may be caused be a genetic disorder, in which a mutation causes the disease, but it may also be that the disorder is caused by a process in which a wild-type protein may be altered to a protein with a loss-of-function or a gain-of-function, which in either possibility may positively influence the course or the disease.

Disclosed is also a method for the deamination of at least one target nucleotide, preferably an adenosine, present in a target RNA molecule in a cell, the method comprising the steps of: providing the cell with an AON or a pharmaceutical composition as disclosed herein; allowing annealing of the AON to the target RNA molecule; and allowing a mammalian enzyme with nucleotide deaminase activity to deaminate the target nucleotide in the target RNA molecule; thereby treating, ameliorating or slowing down progression of a disease.

### BRIEF DESCRIPTION OF THE DRAWINGS

One or more embodiments of the invention will now be described, by way of example only, with reference to the accompanying drawings, in which:

FIG. 1 shows the percentage editing of an endogenous Actin B transcript in human Weri-rb1 cells that were treated with the oligonucleotides RM4000 and RM4875 to RM4898 as indicated. A negative control is shown on the left (Neg).

FIGS. 2A and 2B show the stability difference between RM4000 and RM4887, in two separate studies. RM4000 and RM4887 differ in the sense that RM4000 comprises a methylphosphonate (MP) linkage at linkage position −2, whereas RM4887 instead comprises a mesyl phosphoramidate (PNms) linkage, as disclosed herein, at that position (see FIG. 4). FIG. 2A shows the stability of the AONs in PBS. FIG. 2B shows the stability of the AONs in artificial cerebrospinal fluid (aCSF). Stability was assessed at 5° C., 25° C., and at 40° C., at 3 timepoints: t=0, t=4 weeks, and t=8 weeks.

FIG. 3 shows the editing percentage measured after gymnotic uptake of the 12 AONs as indicated in primary human hepatocytes (PHHs), targeting the c.203A position in the human wildtype SLC10A1 transcript (wherein the A to I editing provides a Q68R change in the encoded NTCP protein; see UK patent applications GB2304438.1 and GB2318087.0, unpublished). One unrelated AONs (RM4777) and a non-treated sample served as negative controls.

FIG. 4 shows the 5′ to 3′ sequences are of AONs RM4875 to RM4898 that, in contrast to RM4000, comprise a PNms linkage at each of the 24 linkage positions, respectively, from the 5′ terminus to the 3′ terminus. The orphan position in these AONs (Cd) is underlined. Also shown are the sequences of RM107361 to RM107385 that were used in an editing experiment (FIG. 3). SEQ ID NO's are indicated. The chemical modifications are as follows: Ae is 2′-MOE modified adenosine; m5Ce is 2′-MOE modified 5-methyl-cytidine; m5Ue is 2′-MOE modified 5-methyl-uridine; Cm, Am, Um, and Gm are 2′-OMe modified cytidine, adenosine, uridine, and guanosine, respectively; Gf, Cf, Af, and Uf are 2′-F modified guanosine, cytidine, adenosine, and uridine, respectively; Zd (the orphan nucleotide in RM107361 to RM107385, underlined) is a deoxynucleotide (deoxycytidine analog) carrying a Benner's base; Id is deoxyinosine; Cd is deoxycytidine; “!” refers to a (1,3-dimethylimidazolidin-2-ylidene) phosphoramidate (PNdmi) linkage; “\hat{ }” refers to a MP linkage; “*” refers to a phosphorothioate (PS) linkage; “#” refers to a PNms linkage; and “^{θ}” refers to a phosphodiester (PO) linkage.

### DETAILED DESCRIPTION

The antisense oligonucleotides as disclosed herein can recruit deaminating enzymes, (such as ADAR1 and/or ADAR2) that are endogenously present in a cell, and can cause RNA editing of a target RNA molecule after it is bound to the target RNA molecule, because the deaminating enzymes are recruited due to the double-stranded nature of the oligonucleotide when bound to the target RNA molecule and the ‘mismatch’ between the target nucleotide and the opposite nucleotide in the antisense oligonucleotide. The oligonucleotides are often abbreviated to “AONs”, but sometimes also referred to as ‘editing oligonucleotides’, or ‘EONs’, even though the RNA editing event is performed by the deamination enzyme and the action of the oligonucleotide only allows the RNA editing to take place. There is a constant need for improving the pharmacokinetic properties of the AONs without negatively affecting the efficiency in which the target adenosine is edited in the target RNA, and/or without negatively affecting the stability of the AON itself, which is constantly prone to breakdown because of nucleases present in a natural cell. Many chemical modifications exist in the generation of AONs (and have been applied in the art), whose properties are not always compatible with the desire of achieving efficient RNA editing. In the search for better pharmacokinetic properties, it was found earlier that a 2′-O-methoxyethyl (or 2′-methoxyethoxy, or 2′-MOE) modification of the ribose of some, but not all, nucleotides surprisingly appeared compatible with efficient ADAR engagement and editing (WO2019/158475). In a similar fashion, it was found earlier that a PS linkage at some, but not all, internucleoside linkages surprisingly appeared compatible with efficient ADAR engagement and editing (WO2019/219581). Also, it was found earlier that phosphonoacetate linkage modifications and/or unlocked nucleic acid (UNA) ribose modifications of some, but not all, positions in the AON appeared compatible with efficient engagement of an enzyme with nucleotide deamination activity and with subsequent deamination (WO2020/165077). Whereas the properties of phosphonoacetate and UNA modifications were known as such, the compatibility thereof with engagement of enzymes with nucleotide deamination activity and with the deamination reaction was not known.

Disclosed herein are AONs that can cause (mediate, bring about, provide, give) RNA editing of a target adenosine in a transcript molecule (pre-mRNA and/or mRNA). The transcript molecule may be of a mutated gene, wherein the mutation causes a disease and wherein the editing can reverse the mutation giving rise to a wildtype protein, or a protein with a wildtype function (for instance when the mutated amino acid is changed to an amino acid that does not cause the disease, or that provides an improved phenotype). The transcript molecule may also be of a wildtype gene, wherein the RNA editing makes that the resulting protein obtains a gain-of-function, which would improve the disease state of the treated subject. The transcript may also be of a wildtype gene, wherein the RNA editing makes that the resulting protein obtains a loss-of-function, which would improve the disease state of the treated subject. Examples are proteins that are mutated but wherein the introduced mutation results in loss of or diminished phosphorylation or loss of or diminished interaction with other cellular moieties that in turn is beneficial in view of the disease that is to be treated. Non-limiting examples of transcripts that may be targeted using the AONs as disclosed herein are SERPINA1 (for the treatment of alpha1-antitrypsin (A1AT) deficiency; see e.g., WO2016/097212, WO2017/220751, WO2018/041973, and WO2021/243023), IDUA (for the treatment of Hurler syndrome; see e.g., WO2017/220751, WO2018/041973, and WO2021/209010), LRRK2 (for the treatment of Parkinson's disease; see e.g., WO2016/097212, WO2017/220751, WO2018/041973, WO2021/231673 and WO2021/242903), ABCA4 (for the treatment of Stargardt disease; see e.g., WO2021/130313 and WO2021/231830), USH2A (for the treatment of Usher syndrome; see e.g., WO2020/157008, WO2020/219981 and WO2021/136404), APP (see e.g., WO2021/113270), CMT1A (see e.g., WO2021/113390), ASS1 (see e.g., WO2021/231675), GJB2 (see e.g., WO2021/231679), MECP2 (for the treatment of Rett syndrome; see e.g., WO2019/071274 and WO2021/231680), OTOF (for the treatment of autosomal recessive non-syndromic hearing loss; see e.g., WO2021/231685 and WO2021/231692), XLRS (see e.g., WO2021/231691), and PCSK9 (for the treatment of hypercholesterolemia; see e.g., WO2023/152371).

Although in a preferred embodiment, the AON as disclosed herein is a single-stranded oligonucleotide comprising an orphan nucleotide opposite the target adenosine, wherein the orphan nucleotide is chemically modified as disclosed herein, and wherein the remainder of the oligonucleotide is chemically modified to prevent it from nuclease breakdown also as disclosed herein, in another embodiment, disclosed is any kind of oligonucleotide or heteroduplex oligonucleotide complex, that may or may not be bound to hairpin structures (internally or at the terminal end(s)), that may be bound to ADAR or catalytic domains thereof, or wherein the oligonucleotide is in a circular format. In a preferred aspect, the AON as disclosed herein is a ‘naked’ oligonucleotide, comprising a variety of chemical modifications in the ribose sugar and/or the base of one or more of the nucleotides within the sequence, that comprises at least one linkage according to the structure of formula (I) as disclosed herein, that can hybridize to the target transcript or a part thereof that includes the target adenosine, and can recruit endogenous (naturally present) ADAR in the target cell for the deamination of the target adenosine. In another embodiment, the AON as disclosed herein, that is delivered in a ‘naked’ form, does not comprise a stem-loop structure for recruitment of the deaminating enzyme, which allows for a shorter AON and improved cellular delivery and trafficking.

Disclosed herein is an antisense oligonucleotide (AON) that is able to mediate adenosine deamination by recruitment of a deaminating enzyme in a cell after the AON has formed a double-stranded complex with a region of a target RNA nucleic acid molecule in a cell, wherein the region comprises a target adenosine, wherein the deaminating enzyme can deaminate the target adenosine into an inosine, and wherein the AON comprises one or more linkage modifications with a structure according to formula (I):

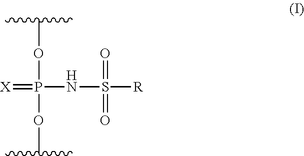

wherein:
X=O or S; and R=an aryl, a substituted aryl, a heterocycle, a substituted heterocycle, an aromatic heterocycle, a substituted aromatic heterocycle, a C₁-C₆ alkoxy, a substituted C₁-C₆ alkoxy, a C₁-C₂₀ alkyl, a substituted C₁-C₂₀ alkyl, a C₁-C₆ alkenyl, a C₁-C₆ substituted alkenyl, a C₁-C₆ alkynyl, a substituted C₁-C₆ alkynyl, or a conjugate group. In a preferred embodiment, X=O and R=methyl.

The deaminating enzyme is preferably an endogenously present (=naturally present) ADAR enzyme in the cell that interacts with the double-stranded complex formed by the AON with the target RNA molecule. The target RNA molecule can be a pre-mRNA or an mRNA molecule and is generally a transcript molecule from a gene. The gene may be mutated or may be wild type. The deamination may result in a loss-of-function of the encoded polypeptide or may result in a gain-of-function of the encoded polypeptide. It may also be that the deamination reverts a stop codon to a UGG codon allowing read-through during translation.

In a preferred aspect, the AON as disclosed herein comprises an internucleoside linkage of the structure of formula (I), wherein X=O and R=CH₃, which linkage is generally referred to herein as a “PNms linkage” (=mesyl phosphoramidate). In other preferred aspects, R equals one of the following structures (a), (b), (c), (d), (e), (f), (g), (h), or (i):

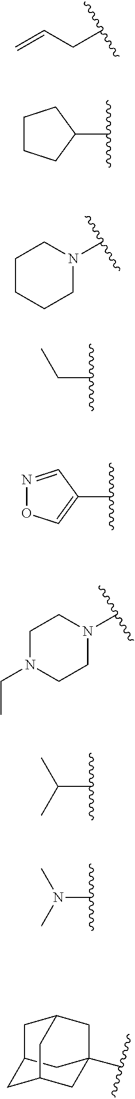

Disclosed herein is also an AON that is able to mediate adenosine deamination by recruitment of a deaminating enzyme in a cell after the AON has formed a double-stranded complex with a region of a target RNA nucleic acid molecule in a cell, wherein the region comprises a target adenosine, wherein the deaminating enzyme can deaminate the target adenosine into an inosine, and wherein the AON comprises a moiety at one and/or both termini with a structure according to formula (II):

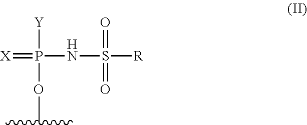

wherein: X=O or S,
Y=O⁻ or S⁻, and
R=an aryl, a substituted aryl, a heterocycle, a substituted heterocycle, an aromatic heterocycle, a substituted aromatic heterocycle, a C₁-C₆ alkoxy, a substituted C₁-C₆ alkoxy, a C₁-C₂₀ alkyl, a substituted C₁-C₂₀ alkyl, a C₁-C₆ alkenyl, a C₁-C₆ substituted alkenyl, a C₁-C₆ alkynyl, a substituted C₁-C₆ alkynyl, or a conjugate group. In a preferred embodiment, X=O and R=methyl.

The AONs as disclosed herein are preferably used for the treatment of a disease in a mammalian subject, preferably a human subject. Hence, in a preferred aspect the cell is a human cell, preferably a human liver, kidney, neuronal or brain cell, depending on the disease to be treated. An AON as disclosed herein is preferably used in a cell wherein the deamination takes place by an endogenously (naturally present) ADAR enzyme which is preferably human ADAR1 or human ADAR2. The disclosure relates to an AON, wherein the nucleotide that is directly opposite the target adenosine is the orphan nucleotide, wherein the internucleoside linkage numbering is such that linkage number 0 is the linkage 5′ from the orphan nucleotide, the linkage positions in the oligonucleotide are positively (+) incremented towards the 5′ end and negatively (−) incremented towards the 3′ end, and wherein the linkage modification according to formula (I) is present at linkage position −2, at the linkage position connecting the 5′ terminal two nucleosides in the AON; and/or at the linkage position connecting the 3′ terminal nucleosides in the AON.

In a preferred aspect, the AON as disclosed herein comprises at least one additional non-naturally occurring chemical modification in the ribose, linkage, or base moiety, with the proviso that the orphan nucleotide, which is the nucleotide in the AON that is directly opposite the target adenosine, is not a nucleotide carrying a non-modified cytosine and wherein it comprises a 2′-OMe ribose substitution. In a preferred aspect, the additional modification in the AON as disclosed herein is an internucleoside linkage modification selected from the group consisting of: PS, phosphonoacetate, phosphorodithioate, MP, phosphoryl guanidine, phosphoramidate, sulfonylphosphoramidate, and PNdmi internucleotide linkage. In a preferred aspect, the additional modification in the AON as disclosed is in the ribose moiety and is a mono- or di-substitution at the 2′, 3′ and/or 5′ position of the ribose, each independently selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy.

Also disclosed is a pharmaceutical composition comprising an AON as disclosed herein, and a pharmaceutically acceptable carrier, solvent, or diluent.

Also disclosed is a method for the deamination of a target adenosine, present in a target RNA nucleic acid molecule in a mammalian cell, the method comprising the steps of: (i) providing the cell with an AON as disclosed herein, or a pharmaceutical composition as disclosed herein; (ii) allowing annealing of the AON to the target RNA nucleic acid molecule; (iii) allowing a deaminating enzyme, preferably naturally present in the cell, to deaminate the target adenosine in the target RNA nucleic acid molecule; and (iv) optionally identifying the presence of the deaminated adenosine in the target RNA nucleic acid molecule. In a preferred aspect, step (iv) comprises: (a) sequencing a region of the target RNA molecule, wherein the region comprises the deaminated target nucleotide; (b) assessing the presence of a functional, elongated, full length and/or wild type protein when the target nucleotide is an adenosine located in a UGA or UAG stop codon; (c) assessing, when the target RNA molecule is pre-mRNA, whether splicing of the pre-mRNA was altered by the deamination; or (d) using a functional read-out, wherein the target RNA molecule after the deamination encodes a functional, full length, elongated and/or wild type protein. In a preferred aspect, the method as disclosed herein further comprises the step of administering to the subject a therapeutically effective dose of a saponin, preferably the triterpene glycoside AG1856, which step may take place before, during, or after providing the AON.

In one embodiment, the AON as disclosed herein, which AON is capable of causing the deamination of a target adenosine in a target RNA transcript molecule by recruitment of an endogenous ADAR enzyme when bound to the target RNA molecule comprises at least one linkage according to the structure of formula (I), and besides that optionally also one or two terminal moieties according to the structure of formula (II) as described herein.

Internucleoside linkages that may be used in the AONs as disclosed herein are those that are disclosed in WO2023/278589.

A preferred internucleoside linkage modification that is used in the AON as disclosed herein has the structure of formula (III):

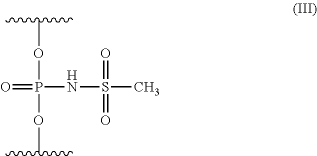 [SMILES: *OP(=O)(NS(C)(=O)=O)O*]

which is also referred to as a mesyl phosphoramidate linkage or a PNms linkage in brief. It is noted that PNms linkages for use in oligonucleotides and as replacement of PS linkages was described earlier (Chelobanov B P et al. Russian J Bioorganic Chemistry. 2017. 43(6):664-668; DOI: 10.1134/S1068162017060024; Klabenkova K et al. Molecules. 2021. 26(17):5420; Miroshnichenko S K et al. Proc Natl Acad Sci USA. 2019. 116(4):1229-1234), and for instance in oligonucleotides that may provide splice switching (Hammond S M et al. Nucleic Acid Ther. 31(3):190-200). However, the use of PNms linkages in AONs that interact with ADAR enzymes and that through this interaction can mediate the enzymatic process referred herein as ‘RNA editing’ was not envisioned.

The one or more PN linkages as depicted in formula (I), present in an AON as disclosed herein, can be independently of each other of R_{P} or S_{P} chirality, or stereorandom.

The one or more PN linkages as depicted in formula (I), in an AON as disclosed herein, can be in tautomeric and/or pH-dependent (de)protonated form, including but not limited to the structures (A), (B), (C), (D), and (E):

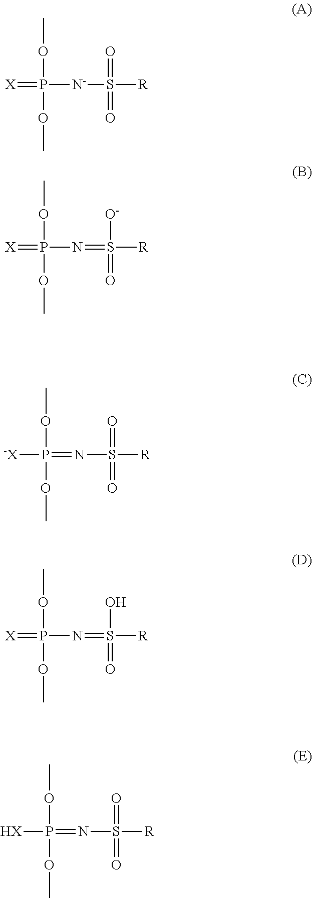

wherein X and R are as indicated above for formula (I).

In one embodiment, an AON as disclosed herein comprises, besides this at least one linkage as disclosed herein, at least one nucleotide comprising one or more non-naturally occurring chemical modifications in the ribose, other linkages, or base moiety, with the proviso that the orphan nucleotide is not a nucleotide with a non-modified cytosine nucleobase and comprising a 2′-OMe substitution in the ribose sugar. In an embodiment, the one or more additional linkage modifications are each independently selected from a PS, phosphonoacetate, phosphorodithioate, MP, sulfonylphosphoramidate, phosphoryl guanidine, or a PNdmi internucleoside linkage (see below). A preferred modified internucleoside linkage is a PS linkage. In one embodiment, all internucleoside linkages of the AON, besides the one or more linkages according to the structure of formula (I), are modified internucleoside linkages. In one embodiment, the AON comprises a PNdmi linkage according to the structure of formula (IV):

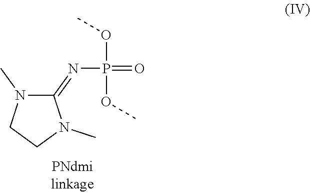

Preferably, the one or more additional modifications in the ribose moiety is a mono- or di-substitution at the 2′, 3′ and/or 5′ position of the ribose, each independently selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy. In an embodiment, the AON comprises one or more mismatches, wobbles, or bulges, wherein a single mismatch may be present when the target adenosine has (for example) an opposite cytidine in the AON. If the orphan nucleotide is a cytidine, that cytidine does not comprise a 2′-OMe ribose substitution.

Disclosed herein is a pharmaceutical composition comprising an AON as described, and a pharmaceutically acceptable carrier, diluent, or solvent.

### Definitions

The term ‘nucleoside’ refers to the nucleobase linked to the (deoxy)ribosyl sugar, without phosphate groups. A ‘nucleotide’ is composed of a nucleoside and one or more phosphate groups. The term ‘nucleotide’ thus refers to the respective nucleobase-(deoxy)ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group), an unlocked nucleic acid (UNA), a threose nucleic acid (TNA), a nucleotide including a linker comprising a phosphodiester, phosphonoacetate, phosphotriester, PS, phosphoro(di)thioate, MP, methyl thiophosphonate, phosphoramidate linkages, PNdmi, and a PNms linkage as described herein.

Sometimes the terms nucleobase, nucleoside and nucleotide are used interchangeably, unless the context clearly requires differently, for instance when a nucleoside is linked to a neighbouring nucleoside and the linkage between these nucleosides is modified. As stated herein, a nucleotide is a nucleoside plus one or more phosphate groups. The terms ‘ribonucleoside’ and ‘deoxyribonucleoside’, or ‘ribose’ and ‘deoxyribose’ are as used in the art.

Sometimes the terms adenosine and adenine, guanosine and guanine, cytidine and cytosine, uracil and uridine, thymine and thymidine/uridine, inosine, and hypoxanthine, are used interchangeably to refer to the corresponding nucleobase on the one hand, and the nucleoside or nucleotide on the other. Thymine (T) is also known as 5-methyluracil (m⁵U) and is a uracil (U) derivative; thymine, 5-methyluracil and uracil can be interchanged throughout the document text. Likewise, thymidine is also known as 5-methyluridine and is a uridine derivative; thymidine, 5-methyluridine and uridine can be interchanged throughout the document text.

Whenever reference is made to an oligonucleotide, oligo, ON, ASO, oligonucleotide composition, antisense oligonucleotide, AON, (RNA) editing oligonucleotide, EON, and RNA (antisense) oligonucleotide, both oligoribonucleotides and deoxyoligoribonucleotides are meant unless the context dictates otherwise. Potentially the oligonucleotide may completely lack RNA or DNA nucleotides (as they appear in nature) and may consist completely of modified nucleotides. Whenever reference is made to an ‘oligoribonucleotide’ it may comprise the bases A, G, C, U, or I. Whenever reference is made to a ‘deoxyoligoribonucleotide’ it may comprise the bases A, G, C, T, or I. However, an AON as disclosed herein may comprise a mix of ribonucleosides and deoxyribonucleosides. When a deoxyribonucleotide is used, hence without a modification at the 2′ position of the sugar, the nucleotide is often abbreviated to Ad, Cd, Gd or T in which the ‘d’ represents the deoxy nature of the nucleoside, while a ribonucleoside that is either normal RNA or modified at the 2′ position is often abbreviated without the ‘d’, and often abbreviated with their respective modifications and as explained herein.

Whenever reference is made to nucleotides in the oligonucleotide, such as cytosine, 5-methylcytosine, 5-hydroxymethylcytosine, 5-formylcytosine, 5-acetylcytosine, 5-hydroxycytosine, and β-D-glucosyl-5-hydroxymethylcytosine are included. Whenever reference is made to adenine, N6-methyladenine, 8-oxo-adenine, 2,6-diaminopurine and 7-methyladenine are included. Whenever reference is made to uracil, dihydrouracil, isouracil, N3-glycosylated uracil, pseudouracil, 5-methyluracil, N1-methylpseudouracil, 4-thiouracil and 5-hydroxymethyluracil are included. Whenever reference is made to guanine, 1-methylguanine, 7-methylguanosine, N2,N2-dimethylguanosine, N2,N2,7-trimethylguanosine and N2,7-dimethylguanosine are included. Whenever reference is made to nucleosides or nucleotides, ribofuranose derivatives, such as 2′-deoxy, 2′-hydroxy, and 2′-O-substituted variants, such as 2′-OMe, are included, as well as other modifications, including 2′-4′ bridged variants. Whenever reference is made to oligonucleotides, one or more linkages is a PNms linkage, whereas the remaining linkages (when not all linkages are PNms) between two mononucleotides may be phosphodiester linkages as well as modifications thereof, including, phosphonoacetate, phosphotriester, PS, phosphoro(di)thioate, MP, phosphoramidate linkages, phosphoryl guanidine, thiophosphoryl guanidine, sulfono phosphoramidate, PNdmi and the like.

The term ‘comprising’ encompasses ‘including’ as well as ‘consisting of’, e.g., a composition ‘comprising X’ may consist exclusively of X or may include something additional, e.g., X+Y. The term ‘about’ in relation to a numerical value x is optional and means, e.g., x±10%. The term ‘conducive to’ can be used interchangeably with ‘capable of facilitating’. When used in the context of an AON that is conducive to ADAR mediated editing, this means that the AON, after entry into the cell, interacts with the target RNA sequence, thereby forming a double stranded structure which is recognized by the ADAR enzyme, which can then deaminate the target adenosine. Hence, the AON itself does not have the enzymatic function (the ADAR enzyme has), but it can induce, cause, organize, produce, facilitate, result in RNA editing after binding to the target RNA molecule.

The word ‘substantially’ does not exclude ‘completely’, e.g., a composition which is ‘substantially free from Y’ may be completely free from Y. Where relevant, the word ‘substantially’ may be omitted from the definition of the invention.

The term ‘complementary’ as used herein refers to the fact that the AON hybridizes under physiological conditions to a second nucleic acid strand (for instance when the oligonucleotide as a first nucleic acid strand (also sometimes referred to as a ‘guide oligonucleotide’) forms a heteroduplex RNA editing oligonucleotide complex, or HEON, with another complementary nucleic acid strand), or when it forms a double stranded complex with the target RNA molecule. The term does not necessarily mean that each nucleotide in a nucleic acid strand has a perfect pairing with its opposite nucleotide in the opposite sequence. In other words, while an AON may be complementary to a target sequence, there may be mismatches, wobbles and/or bulges between the oligonucleotide and the target sequence, while under physiological conditions that AON still hybridizes to the target sequence such that the cellular RNA editing enzymes can deaminate the target adenosine to an inosine. The term ‘substantially complementary’ therefore also means that despite the presence of the mismatches, wobbles, and/or bulges, the AON has enough matching nucleotides with the target sequence that under physiological conditions the AON hybridizes to the target RNA molecule. As shown herein, an AON may be complementary, but may also comprise one or more mismatches, wobbles and/or bulges with the target sequence, if under physiological conditions the AON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that most stable interactions between probe and target are where the probe and target have at least 70%, preferably at least 80%, more preferably at least 90% sequence identity.

The term ‘mismatch’ is used herein to refer to opposing nucleotides in a double stranded RNA complex which do not form perfect base pairs according to the Watson-Crick base pairing rules. In the historical sense, mismatched nucleotides are G-A, C-A, U-C, A-A, G-G, C-C, U-U pairs. In some embodiments AONs as disclosed herein comprise fewer than four mismatches with the target sequence, for example 0, 1 or 2 mismatches. ‘Wobble’ base pairs are G-U, I-U, I-A, and I-C base pairs. Although a G:G pairing would be considered a mismatch, that does not necessarily mean that the interaction is unstable, which means that the term ‘mismatch’ may be somewhat outdated based on the current disclosure where a Hoogsteen base-pairing may be seen as a mismatch based on the origin of the nucleotide but still be relatively stable. An isolated G:G pairing in duplex RNA can for instance be quite stable, but still be defined as a mismatch.

The term ‘splice mutation’ relates to a mutation in a gene that encodes for a pre-mRNA, wherein the splicing machinery is dysfunctional in the sense that splicing of introns from exons is disturbed and due to the aberrant splicing, the subsequent translation is out of frame resulting in premature termination of the encoded protein. Often such shortened proteins are degraded rapidly and do not have any functional activity.

An AON (and the complementary nucleic acid strand when two oligonucleotides form a heteroduplex RNA editing oligonucleotide, which is sometimes referred to as a “HEON”) as disclosed herein may be chemically modified almost in its entirety, for example by providing nucleotides with a ribose sugar moiety carrying a 2′-OMe substitution, a 2′-F substitution, or a 2′-0-methoxyethyl (2′-MOE) substitution.

The orphan nucleotide in the AON is preferably a cytidine or analog thereof (such as a nucleotide carrying a Benner's base) or a uridine or analog thereof (such as iso-uridine). The orphan nucleotide preferably comprises a deoxyribose (2′-H; =DNA) but may also comprise a diF modification at the 2′ position of the sugar. In an embodiment at least one and in another embodiment both the neighbouring (directly adjacent) nucleotides flanking the orphan nucleotide do not comprise a 2′-OMe modification. Complete modification wherein all nucleotides of the oligonucleotide hold a 2′-OMe modification (including the orphan nucleotide), with natural bases, results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-OMe group (at least when there are no other chemical substitutions or modifications within the nucleotide), or by providing a guanine or adenine as opposing base, as these two nucleobases are also able to reduce editing of the opposing adenosine.

Various chemistries and modifications are known in the field of oligonucleotides that can be readily used in accordance with the invention. The regular internucleoside linkages between the nucleotides may be altered by mono- or di-thioation of the phosphodiester bonds to yield PS esters or phosphorodithioate esters, respectively. Other modifications of the internucleoside linkages are possible, including amidation and peptide linkers.

In an embodiment, the AON as disclosed herein comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides. When the AON is to be delivered as is, without a vector, also referred to as a ‘naked form’, the length of the AON is preferably limited to 15 to 60 nucleotides to reduce the risk of degradation. Furthermore, in a naked form, the AON is preferably chemically modified as outlined herein to lower the risk of degradation.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of human ADAR to deaminate adenosines in a non-discriminative manner, reacting with any adenosine it encounters. The specificity of hADAR1 and 2 can be increased by introducing chemical modifications and/or ensuring several mismatches in the dsRNA, which presumably helps to position the dsRNA binding domains in a way that has not been clearly defined yet. Additionally, the deamination reaction itself can be enhanced by providing an oligonucleotide that comprises a mismatch opposite the adenosine to be edited. Following the instructions in the present application, those of skill in the art will be capable of designing the complementary portion of the oligonucleotide according to their needs.

It will be understood by a person having ordinary skill in the art that the extent to which the editing enzymes inside the cell are redirected to other target sites may be regulated by varying the affinity of the first nucleic acid strand for the recognition domain of the editing enzyme. The exact modification may be determined through some trial and error and/or through computational methods based on structural interactions between the AON and the recognition domain of the editing enzyme. In addition, or alternatively, the degree of recruiting and redirecting the editing enzyme resident in the cell may be regulated by the dosing and the dosing regimen of the AON. This is something to be determined by the experimenter (in vitro) or the clinician, usually in phase I and/or II clinical trials.

Disclosed herein is the site-specific editing of target adenosines in RNA sequences in eukaryotic, preferably metazoan, more preferably mammalian, even more preferably human cells, and most preferably human liver cells such as hepatocytes, human neuronal cells, human kidney cells or human cells in adipose tissue. The target cell can be located in vitro, ex vivo or in vivo. One advantage of the AON as disclosed herein is that it can be used with cells in situ in a living organism, but it can also be used with cells in culture. In some embodiments cells are treated ex vivo and are then introduced into a living organism (e.g., re-introduced into an organism from whom they were originally derived). The AON as disclosed herein can also be used to edit target RNA sequences in cells from a transplant or within a so-called organoid, e.g., a liver tissue organoid. Organoids can be thought of as three-dimensional in vitro-derived tissues but are driven using specific conditions to generate individual, isolated tissues. In a therapeutic setting they are useful because they can be derived in vitro from a patient's cells, and the organoids can then be re-introduced to the patient as autologous material which is less likely to be rejected than a normal transplant.

Without wishing to be bound by theory, the RNA editing through human ADAR2 for example is thought to take place on primary transcripts in the nucleus, during transcription or splicing, or in the cytoplasm, where e.g., mature mRNA, miRNA or ncRNA can be edited. Generally spoken, RNA editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether to prevent, delay or treat disease or for any other purpose, in medicine or biotechnology, as diagnostic, prophylactic, therapeutic, research tool or otherwise, are encompassed by the present disclosure.

The amount of AON to be administered, the dosage and the dosing regimen can vary from cell type to cell type, the disease to be treated, the target population, the mode of administration (e.g., systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change, or a change in (the level of, or activity of) a specified biomarker (such as plasma levels of LDL-C, or plasma bile acid levels, for example). It is possible that higher doses of AONs could compete for binding to an ADAR enzyme within a cell, thereby depleting the amount of the enzyme, which is free to take part in RNA editing, but routine dosing trials will reveal any such effects for a given AON and a given target.

One suitable trial technique involves delivering the AON to cell lines, or a test organism and then taking biopsy samples at various time points thereafter. The sequence of the target RNA can be assessed in the biopsy sample and the proportion of cells having the modification can easily be followed. As mentioned above, plasma level concentrations of LDL-C and/or triglycerides in a sample from a treated subject is a proper biomarker for assessing the function of certain proteins in the subject, before and after treatment, or with or without treating the subject with an AON as disclosed herein. After this trial has been performed once then the knowledge can be retained, and future delivery can be performed without needing to take biopsy samples. A method as disclosed herein can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively, as indicated above, the change may be assessed on the function of the protein before, during, and/or after treatment or assessing any other potential marker, which measurements are preferably performed in vitro on samples obtained from the treated subject.

After RNA editing has occurred in a cell, the modified RNA can become diluted over time, for example due to cell division, limited half-life of the edited RNAs, etc. Thus, in practical therapeutic terms a method as disclosed herein may involve repeated delivery of an AON until enough target RNAs have been modified to provide a tangible benefit to the patient and/or to maintain the benefits over time.

AONs as disclosed herein are particularly suitable for therapeutic use, and so disclosed is also a pharmaceutical composition comprising an AON as disclosed herein and a pharmaceutically acceptable carrier, solvent, or diluent. In some embodiments the pharmaceutically acceptable carrier can simply be a saline solution. This can usefully be isotonic or hypotonic, particularly for pulmonary delivery. Disclosed is also a delivery device (e.g., syringe, inhaler, nebuliser) which includes a pharmaceutical composition as disclosed herein. Disclosed is also a route of delivery, such intramuscular delivery, intranasal delivery, subcutaneous delivery by direct injection or otherwise, which delivery method depends on the disease to be treated and the best mode of action.

In one embodiment, depending on the ultimate deamination effect of A to I conversion, the identification step of whether the editing has taken place, comprises the following steps: sequencing the target RNA; assessing the presence or absence of a non-, or less-functional protein; assessing whether splicing of the pre-mRNA was altered by the deamination; or using a functional read-out, because the target RNA after the deamination should encode a protein with a lower or absent functionality, or on the other hand, an increased or regained functionality. The identification of the deamination into inosine may be a functional read-out using a suitable biomarker. A functional assessment will generally be according to methods known to the skilled person. A suitable manner to identify the presence of an inosine after deamination of the target adenosine is of course dPCR or even sequencing, using methods that are well-known to the person skilled in the art. However, the person skilled in the art of liver disease will preferably apply tests to monitor certain biomarkers related to liver function(s).

The AON as disclosed herein is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g., through nebulization), intranasally, orally, by injection or infusion, intravenously, subcutaneously, intradermally, intramuscularly, intra-tracheally, intra-peritoneally, intrarectally, intrathecally, intra-cisterna magna, parenterally, and the like. Administration may be in solid form, in the form of a powder, a pill, a gel, a solution, a slow-release formulation, or in any other form compatible with pharmaceutical use in humans.

In one embodiment, a method as disclosed herein comprises the steps of administering to the subject an AON or pharmaceutical composition as disclosed herein, allowing the formation of a double stranded nucleic acid complex of the AON with its specific complementary target nucleic acid molecule in a cell in the subject; allowing the engagement of an endogenous present adenosine deaminating enzyme, such as ADAR 1 or ADAR2; and allowing the enzyme to deaminate the target adenosine in the target nucleic target molecule to an inosine, thereby alleviating, treating, ameliorating, or slowing down progression of the disease.

RNA editing molecules present in the cell will usually be proteinaceous in nature, such as the ADAR enzymes found in metazoans, including mammals. The ones of most interest are the human ADARs, hADAR1 and hADAR2, including any isoforms thereof. RNA editing enzymes known in the art, for which oligonucleotide constructs as disclosed herein may conveniently be designed, include the adenosine deaminases acting on RNA (ADARs), such as hADAR1 and hADAR2 in humans or human cells and cytidine deaminases. It is known that hADAR1 exists in two isoforms; a long 150 kDa interferon inducible version and a shorter, 110 kDa version, that is produced through alternative splicing from a common pre-mRNA. Consequently, the level of the 150 kDa isoform available in the cell may be influenced by interferon, particularly interferon-gamma (IFN-γ). hADAR1 is also inducible by TNF-α. This provides an opportunity to develop combination therapy, whereby IFN-γ or TNF-α and AONs as disclosed herein are administered to a patient either as a combination product, or as separate products, either simultaneously or subsequently, in any order. Certain disease conditions may already coincide with increased IFN-γ or TNF-α levels in certain tissues of a patient, creating further opportunities to make editing more specific for diseased tissues. It will be understood by a person having ordinary skill in the art that the extent to which the editing entities inside the cell are redirected to other target sites may be regulated by varying the affinity of the first nucleic acid strand for the recognition domain of the editing molecule.

### Chemical Modifications

All chemical modifications listed herein that may be used in the AON as disclosed herein may also be used for a sense strand that is complementary to the AON, when the AON and the complementary strand form a so-called heteroduplex RNA editing oligonucleotide (HEON) complex, such as described in PCT/EP2023/079290 (unpublished), except that the opposite sense strand does not have an orphan nucleotide. Hence, the modification related to the orphan nucleotide relate only to the AON as disclosed herein, but all other modifications relate to the AON as disclosed herein and any (protecting) sense oligonucleotide that may be used together with the AON in a pharmaceutical product. This includes the use of hydrophobic moieties (such as tocopherol and cholesterol) and cell-specific ligands (such as GalNAc moieties), that have also been described herein, and in detail in PCT/EP2023/079290 (unpublished), which may either be bound to the AON or its opposite strand, or both. Preferred GalNAc moieties that can be used in the context of the AONs as disclosed herein are disclosed in WO2022/271806.

The AON as disclosed herein may comprise one or more naturally occurring internucleoside linkages. The AON as disclosed herein does comprise at least one internucleoside linkage modification according to the structure of formula (I), preferably wherein X=O and R=CH₃. Further linkage modifications, including those of the structure of formula (I) may be present in the AON, with or without other available linkage modifications, as outlined herein. The choice and number of modified linkages may depend on the specific target, the sequence, the length, and the stability of the AON observed in a particular cell type of interest, which can be assessed by methods known to the person skilled in the art.

In one embodiment, at least one, at least two, at least three, or at least four internucleoside linkages from the 5′ and/or the 3′ terminus of the AON are modified internucleoside linkages. An internucleoside linkage that is often used in AONs used in RNA editing is a PS linkage, which can be present in the AON whereas elsewhere in the AON the one or more linkages are present according to the structure of formula (I), preferably wherein that one or more linkages are PNms linkages as those reduce the toxicity of the AON compared to an AON with PS linkages on those positions, and as shown in the present disclosure provide an increased stability to the AON. In one embodiment, the AON is 20, 21, 22, 23, 24, 25, 26, 27, 28, 29, 30, 31, 32, 33, 34, or 35 nucleotides in length and comprises 1, 2, 3, 4, 5, 6, 7, or 8 PNms linkages. In another embodiment, the AON does not comprise MP linkages, but instead comprises one or more PNms linkages, such as on linkage position −2. In another embodiment, the AON does not comprise PNdmi linkages, but instead comprises 2, 3, or more PNms linkages, such as at the two terminal linkage positions (on the 5′ and 3′ ends). In another embodiment, the AON does not comprise MP and PNdmi linkages, but comprises only linkages selected from PO, PS, and PNms, with the proviso that the AON comprises at least 1, 2, or 3 PNms linkages and at least 1 PS linkage. In one embodiment, all internucleoside linkages of the AON are non-naturally modified internucleoside linkages and the AON does not comprise PO linkages. In one embodiment, the AON does not comprise a MP linkage. In one embodiment, the AON comprises at least one PNdmi linkage, preferably linking the most terminal two nucleosides at the 5′ and/or 3′ end of the AON. A PNdmi linkage as preferably used in the AONs as disclosed herein has the structure of formula (IV). In one embodiment, the AON does not comprise a PNdmi linkage and does not comprise a MP linkage. In one embodiment, the linkage at linkage position −2 is a PNms linkage. In one embodiment, the linkage connecting the two nucleosides at the 5′ terminus of the AON is a PNms linkage. In one embodiment, the linkage connecting the two nucleosides at the 3′ terminus of the AON is a PNms linkage. In one embodiment, the linkage connecting the two nucleosides at the 5′ terminus of the AON and the linkage connecting the two nucleosides at the 3′ terminus of the AON are both PNms linkages. In one embodiment, the linkage connecting the two nucleosides at the 5′ terminus of the AON, the linkage connecting the two nucleosides at the 3′ terminus of the AON, and the linkage at linkage position −2 are all PNms linkages. In one embodiment, the linkage connecting the two nucleosides at the 5′ terminus of the AON and the linkage at linkage position −2 are both PNms linkages. In one embodiment, the linkage connecting the two nucleosides at the 3′ terminus of the AON and the linkage at linkage position −2 are both PNms linkages. The present disclosure also relates to a method for increasing the stability of an AON that can mediate RNA editing through recruiting an endogenous ADAR enzyme when forming a double stranded complex with a target transcript molecule, by introducing into the AON a PNms linkage, preferably instead of an MP linkage. In another preferred aspect, PNms linkages are applied instead of PO, PS, and/or PNdmi linkages. Other internucleoside linkages that may be used in the AONs as disclosed herein are those that are disclosed in WO2023/278589.

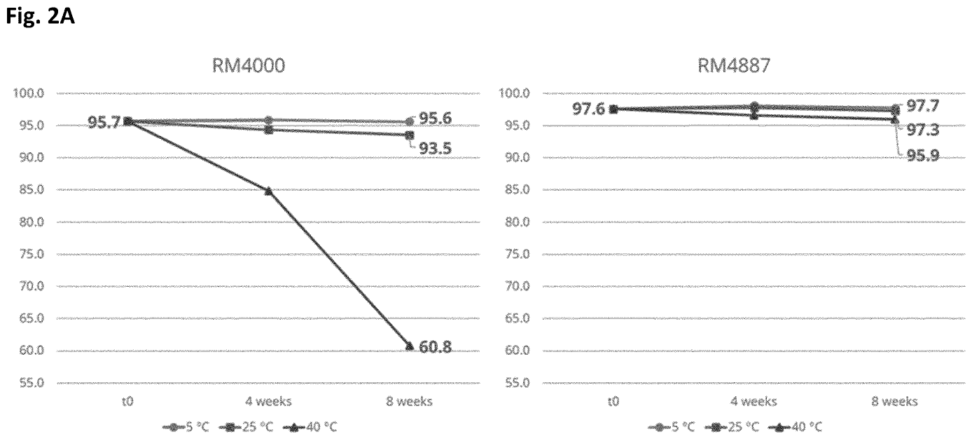

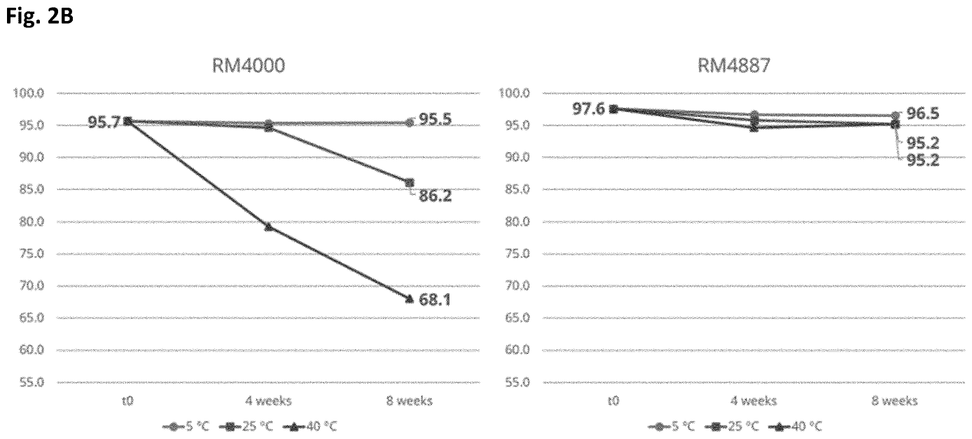

A common limiting factor in oligonucleotide-based therapies are the oligonucleotide's ability to be taken up by the cell (when delivered per se, or ‘naked’ without applying a delivery vehicle), its biodistribution and its resistance to nuclease-mediated breakdown. The skilled person is aware, and it has been described in detail in the art, that a variety of chemical modifications can assist in overcoming such limitations. Examples of such now commonly used chemical modifications are the 2′-O-methyl (often abbreviated to 2′-OMe or 2′-O-Me), 2′-F and 2′-O-methoxyethyl (often also referred to as 2′-methoxyethoxy, or 2′-MOE) modifications of the sugar and the use of PS linkages between nucleosides. WO2020/201406 discloses the use of MP linkage modifications at certain positions surrounding the orphan nucleotide in the first nucleic acid strand. Although the presence of MP linkages is compatible with RNA editing by human ADAR enzymes, introducing MP linkages during the manufacturing of oligonucleotides is challenging in view of additional manufacturing (purification) steps in the coupling and decoupling process (data not shown). Hence, in a preferred embodiment, the position where an MP linkage is preferred for an AON to function properly, is occupied by a linkage modification according to the structure of formula (I), not only for proper functioning of the AON to produce RNA editing, but also to solve the problems that exist in chemically introducing MP linkages. Moreover, as disclosed herein, the introduction of a linkage according to formula (I) at this linkage position −2 in the AON (instead of an MP linkage) increases the stability of the AON in a very significant manner (see FIGS. 2A and 2B). This does not rule out the possibility and potential need to introduce a linkage according to the structure of formula (I) at other (and multiple) positions within the AON, such as for example the 5′ and 3′ terminal linkage positions (for instance thereby replacing the PNdmi linkage that is often present there). Nevertheless, it is explicitly not excluded that an AON as disclosed herein comprises at least one internucleoside linkage according to the structure of formula (I), and/or at least one internucleoside linkage that is MP, and/or at least one internucleoside linkage that is PNdmi.

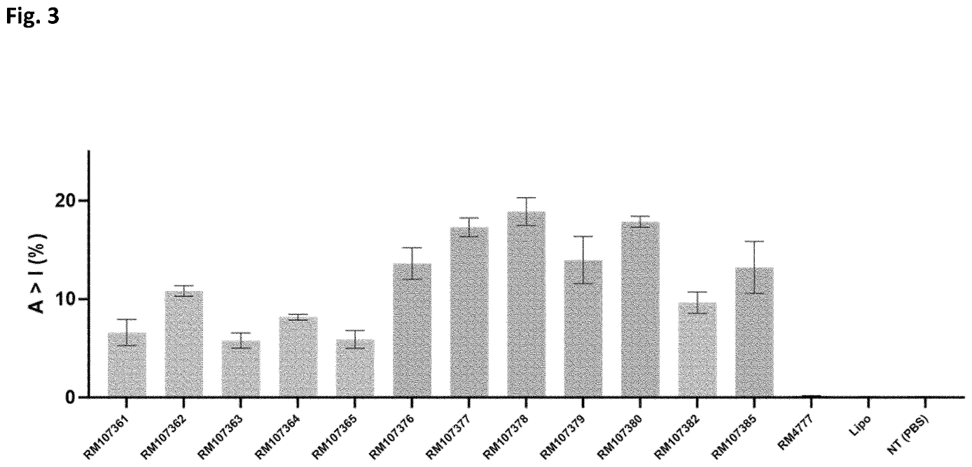

As shown in the present disclosure it was surprisingly found that by introducing a PNms linkage at certain positions in an AON that can cause RNA editing by recruiting endogenous ADAR enzymes, for the targeted deamination of specific adenosines in a target (pre-) mRNA transcript molecule (thereby replacing a PS linkage), RNA editing efficiency increases. For instance, as outlined in detail in the accompanying examples, the editing percentages increased when a PNms linkage was present at linkage positions +5, +4, +3, +2, +1, 0, or −1 (RM4880, RM4881, RM4882, RM4883, RM4884, RM4885, and RM4886, respectively). Notably, in the benchmark AON (RM4000) an MP linkage is present at linkage position -2, and PNdmi linkages are present at the 5′ and 3′ terminal linkages. Replacing the PNdmi linkages at the 5′ side (linkage position +10, see RM4875) and replacing the PNdmi linkage at the 3′ side (linkage position -13, see RM4898) resulted in editing efficiencies that were comparable to what was found with RM4000, indicating that PNms can be a proper alternative for PNdmi at those positions. By using a PNms linkage at linkage position −2 (see RM4887) instead of the MP linkage, the editing efficiency remains at a similar level as was found with RM4000, indicating that PNms at this position is in fact a good alternative, especially because it is difficult to manufacture AONs comprising MP linkages. But, very importantly, and quite surprisingly, the stability of RM4887 appeared much higher than RM4000. The accompanying examples and FIG. 2 clearly show that the long-term stability of RM4887 (with the PNms linkage at linkage position -2) is much higher in both PBS and aCSF formulations at elevated temperatures than when a MP linkage is present at that position (RM4000). Strikingly, in an alternative setting (with another target transcript molecule, and wherein deamination of another target adenosine was assessed) an AON comprising a PNdmi linkage at the 5′ and 3′ linkage positions (linkage position +12 and −13 respectively) and further comprising an MP linkage at linkage position -2 (see RM107362) appeared to provide proper editing of the target adenosine (see FIG. 3), reaching levels above 10% in primary human hepatocytes. But quite unexpectedly, when both PNdmi linkages as well as the MP linkage were replaced by PNms (see RM107378), the editing percentage almost doubled. This indicates that PNms is not only a good alternative for PS linkages, or PNdmi linkages, but that when the PNms is present at the terminal linkage positions, as well as on linkage position -2, stability of the AON is increased and editing efficiency goes up.

The ribose 2′ groups in all nucleotides of the AON, except for the ribose sugar moiety of the orphan nucleotide that has certain limitations in respect of compatibility with RNA editing, can be independently selected from 2′-H (i.e., DNA), 2′-OH (i.e., RNA), 2′-OMe, 2′-MOE, 2′-F, or 2′-4′-linked (for instance a locked nucleic acid (LNA)), or other ribosyl 1′-substitutions, 2′ substitutions, 3′ substitutions, 4′ substitutions or 5′ substitutions. The orphan nucleotide in the AON that comprises no other chemical modifications to the ribose sugar, the base, or the linkage preferably does not carry a 2′-OMe or 2′-MOE substitution but may carry a 2′-F, a 2′,2′-difluoro (diF), or 2′-ara-F (FANA) substitution or may be DNA. WO2024/013360 describes the modification of the 2′ position of the ribose sugar moiety of the orphan nucleotide by a 2′,2′-disubstituted substitution such as diF, which is also applicable to what is disclosed here. The 2′-4′ linkage can be selected from many linkers known in the art, such as a methylene linker, amide linker, or constrained ethyl linker (cEt).

The AONs as disclosed herein may also be administered in the context of aids that will increase the entry of the AON into the target cell and/or its endosomal escape as soon as it is in the cell. Moieties that can be applied for such applications are for example a set of chemical compounds (generally purified from nature) referred to as “saponins” or “triterpene glycosides”. A preferred saponin that can be used in the methods as disclosed herein is AG1856, disclosed in WO2021/122998 and further described for use with RNA editing producing oligonucleotides in PCT/EP2024/051278 (unpublished). In a preferred aspect, the AG1856 saponin is conjugated to the AON of the present disclosure, thereby providing the targeted delivery of a stable AON to a particular cell of interest where it can target a target adenosine in a preferred target transcript molecule.

Disclosed here is an AON for use in the deamination of a target adenosine in a target RNA, wherein the AON is complementary to a stretch of nucleotides in the target RNA that includes the target adenosine, wherein the nucleotide in the AON that is directly opposite the target nucleotide is the orphan nucleotide. The orphan nucleotide is preferably a deoxycytidine or a deoxynucleotide carrying a modified base or base analogue with a NH moiety at the position like the ring nitrogen (e.g., Benner's base Z). The nucleotide numbering in the AON is such that the orphan nucleotide is number 0 and the nucleotide 5′ from the orphan nucleotide is number +1. Counting is further positively (+) incremented towards the 5′ end and negatively (−) incremented towards the 3′ end, wherein the first nucleotide 3′ from the orphan nucleotide is number -1. The internucleoside linkage numbering in the AON is such that linkage number 0 is the linkage 5′ from the orphan nucleotide, and the linkage positions in the oligonucleotide are positively (+) incremented towards the 5′ end and negatively (−) incremented towards the 3′ end.

In one embodiment, the AON comprises one or more (chirally pure or chirally mixed) PS linkages. In one embodiment, PS linkages connect the terminal 3, 4, 5, 6, 7, or 8 nucleotides on each end of the AON. In one embodiment, the AON comprises one of more (chirally pure or chirally mixed) phosphoramidate (PN) linkages. In one embodiment, a PN linkage connects the terminal two nucleotides on each end of the AON. This may be PNdmi, but according to the present disclosure, the terminal two nucleotides on each end of the AON may be connected through a PNms linkage.

A nucleoside in the AON may be a natural nucleoside (deoxyribonucleoside or ribonucleoside) or a non-natural nucleoside. It is noted that for RNA editing, in which double-stranded RNA is generally the substrate for enzymes with deamination activity (such as ADARs), ribonucleosides are considered ‘natural’, while deoxyribonucleosides may then be, for the sake of argument, considered as non-natural, or modified, simply because DNA is not present in the RNA-RNA double stranded (natural) substrate configurations. The skilled person appreciates that when the nucleotide has a natural ribose moiety, it may still be non-naturally modified in the base and/or the linkage.

In addition to the specific preferred chemical modifications at certain positions in compounds as disclosed herein, AONs as disclosed herein may comprise one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e., the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-OMe, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino)propyl), 2′-O-(2-amino)ethyl, 2′-O-(2-(dimethylamino)ethyl); 2′-deoxy (DNA); 2′-0-(haloalkyl)methyl such as 2′-O-(2-chloroethoxy)methyl (MCEM), 2′-O-(2,2-dichloroethoxy)methyl (DCEM); 2′-O-alkoxycarbonyl such as 2′-O-[2-(methoxycarbonyl)ethyl](MOCE), 2′-O-[2-N-methylcarbamoyl)ethyl](MCE), 2′-O-[2-(N,N-dimethylcarbamoyl)ethyl](DCME); 2′-halo e.g. 2′-F, FANA; 2′-O-[2-(methylamino)-2-oxoethyl](NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-I-LNA monomer, a β-d-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-0,4′-C) constrained ethyl (cEt) BNA monomer, a (2′-0,4′-C) constrained methoxyethyl (cMOE) BNA monomer, a 2′,4′-BNA^{NC}(NH) monomer, a 2′,4′-BNA^{NC}(NMe) monomer, a 2′,4′-BNA^{NC}(NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-I-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All these modifications are known to the person skilled in the art.

The AON as disclosed herein, in contrast to what has been described for siRNA, or gapmers and their relation towards RNase breakdown and the use of such gapmers in double-stranded complexes (see for instance EP 3954395 A1), does not comprise a stretch of DNA nucleotides which would make a target sequence (or a sense nucleic acid strand) a target for RNase-mediated breakdown. It is not desired that the target transcript molecule is degraded through the binding of the AON to the transcript molecule. In one embodiment, the AON does not comprise four or more consecutive DNA nucleotides anywhere within its sequence. In an embodiment, the AON is composed of as much (chemically) modified nucleotides as possible to enhance the resistance towards RNase-mediated breakdown, while at the same time being as efficient as possible in producing an RNA editing effect. This means that the orphan nucleotide and several other nucleotides within the AON may be DNA, but also that there is no stretch of four or more consecutive DNA nucleotides within the AON. Hence, the AON as disclosed herein is not a gapmer. A gapmer reduces the expression of a target transcript but does not produce RNA editing of a specified adenosine within the target transcript. A gapmer is in principle a single-stranded nucleic acid consisting of a central region (DNA gap region with at least four consecutive deoxyribonucleotides) and wing regions positioned directly at the 5′ end (5′ wing region) and the 3′ end (3′ wing region) thereof. In contrast, the AON as disclosed herein may be any oligonucleotide that produces an RNA editing effect in which a target adenosine in a target RNA molecule is deaminated to an inosine, and accordingly is resistant to RNase-mediated breakdown as much as possible to yield this effect and to allow the mRNA transcript being translated into a protein.

In one embodiment, the AON, or the sense strand to which it may be annealed before entering a target cell, is bound to a hydrophobic moiety, such as palmityl or an analog thereof, cholesterol or analog thereof, or tocopherol or analog thereof. It is preferably bound to the 5′ terminus. In case a hydrophobic moiety is bound to the 5′ terminus as well as to the 3′ terminus, such hydrophobic moieties may the same or different. The hydrophobic moiety bound to the oligonucleotide may be bound directly, or indirectly mediated by another substance. When the hydrophobic moiety is bound directly, it is sufficient if the moiety is bound via a covalent bond, an ionic bond, a hydrogen bond, or the like. When the hydrophobic moiety is bound indirectly, it may be bound via a linking group (a linker). The linker may be a cleavable or an uncleavable linker. A cleavable linker refers to a linker that can be cleaved under physiological conditions, for example, in a cell or an animal body (e.g., a human body). A cleavable linker is selectively cleaved by an endogenous enzyme such as a nuclease, or by physiological circumstances specific to parts of the body or cell, such as pH or reducing environment (such as glutathione concentrations). Examples of a cleavable linker comprise, but is not limited to, an amide, an ester, one or both esters of a phosphodiester, a phosphoester, a carbamate, and a disulfide bond, as well as a natural DNA linker. Cleavable linkers also include self-immolative linkers. An uncleavable linker refers to a linker that is not cleaved under physiological conditions, or very slowly compared to a cleavable linker, for example, in a PS linkage, modified or unmodified deoxyribonucleosides linked by a PS linkage, a spacer connected through a PS bond and a linker consisting of modified or unmodified ribonucleosides. There is no restriction on the chain length, when a linker is a nucleic acid such as DNA, or an oligonucleotide. However, it may be usually from 2 to 20 bases in length, from 3 to 10 bases in length, or from 4 to 6 bases in length. There is no restriction on the length or composition of a spacer that is connects the ligand and the oligonucleotide, and may include for example ethylene glycol, triethylene glycol (TEG), HEG, alkyl chains, propyl, 6-aminohexyl, or dodecyl. In one embodiment, a GalNAc moiety is bound to the AON as disclosed herein via a TEG linker. One or more other types of molecules may be bound to the AON through one or more linkers, including peptides, sugars, vitamins, polymers, aptamers, (fragments of) antibodies, small molecules, and the like.

Disclosed herein is also a pharmaceutical composition comprising the AON as disclosed herein, and further comprising a pharmaceutically acceptable carrier, solvent, diluent, and/or other additive (such as a saponin or triterpene glycoside like AG1856 (as discussed above), which in fact may also be administered separately from the AON) and may be dissolved in a pharmaceutically acceptable organic solvent, or the like. Dosage forms in which the AON or the pharmaceutical composition are administered may depend on the disorder to be treated and the tissue that needs to be targeted and can be selected according to common procedures in the art. The pharmaceutical compositions may be administered by a single-dose administration or by multiple dose administration. It may be administered daily or at appropriate time intervals, which may be determined using common general knowledge in the field and may be adjusted based on the disorder and the efficacy of the active ingredient.

In one embodiment, the AON comprises at least one internucleoside linkage according to the structure of formula (I), and the AON further comprises at least one nucleotide with a sugar moiety that comprises a 2′-OMe modification, and/or the AON comprises at least one nucleotide with a sugar moiety that comprises a 2′-MOE modification, and/or the AON comprises at least one nucleotide with a sugar moiety that comprises a 2′-F modification, and/or the orphan nucleotide carries a 2′-H in the sugar moiety and is therefore referred to as a DNA nucleotide, even though additional modifications may exist in its base and/or linkage to its neighbouring nucleosides. In one embodiment, the orphan nucleotide carries a 2′-F in the sugar moiety. In one embodiment, the orphan nucleotide carries a diF substitution in the sugar moiety. In one embodiment, the orphan nucleotide carries a 2′-F and a 2′-C-methyl in the sugar moiety. In one embodiment, the orphan nucleotide comprises a 2′-F in the arabinose configuration (FANA) in the sugar moiety. In one embodiment, the AON is an antisense oligonucleotide that can form a double stranded nucleic acid complex with a target RNA molecule, wherein the double stranded nucleic acid complex can recruit an adenosine deaminating enzyme for deamination of a target adenosine in the target RNA molecule, wherein the nucleotide in the AON that is opposite the target adenosine is the orphan nucleotide, and wherein the orphan nucleotide has the structure of formula (V):

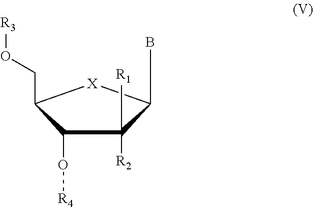

wherein: X is O, NH, OCH₂, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-3-yl-2(1H)-pyridone; R₁ and R₂ are both selected, independently, from H, OH, F or CH₃; R₃ is the part of the AON that is 5′ of the orphan nucleotide, consisting of 7 to 30 nucleotides; and R₄ is the part of the AON that is 3′ of the orphan nucleotide, consisting of 4 to 25 nucleotides. The nucleotide 3′ and/or 5′ from the orphan nucleotide may be DNA, more preferably the nucleotide at the 3′ (position -1).

In one embodiment, the AON comprises at least one MP internucleoside linkage according to the structure of formula (VI):

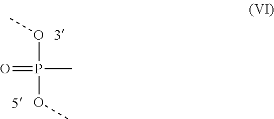

As was noted in the art (WO2020/201406), a suitable position for an MP linkage in an AON is linkage position -2, thereby connecting the nucleoside at nucleotide position -1 with the nucleoside at nucleotide position -2. In a preferred embodiment, this linkage position, in an AON as disclosed herein, comprises a linkage modification according to the structure of formula (I), instead of an MP linkage.

In one embodiment, the AON comprises at least one nucleotide with a sugar moiety that comprises a 2′-fluoro (2′-F) modification. A preferred position for the nucleotide that carries a 2′-F modification is position -3 in AON, which may be present together with an identical 2′ modification in the orphan nucleotide as discussed above.

In one embodiment, the AON comprises at least one phosphonoacetate and/or at least one phosphonoacetamide internucleoside linkage.

In one embodiment, the AON comprises at least one nucleotide comprising a locked nucleic acid (LNA) ribose modification, or an unlocked nucleic acid (UNA) ribose modification. In an embodiment, the AON comprises at least one nucleotide comprising a threose nucleic acid (TNA) ribose modification.

The skilled person knows that an oligonucleotide, such as an AON as outlined herein, generally consists of repeating monomers. Such a monomer is most often a nucleotide or a chemically modified nucleotide. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide.

A modification in the pentose sugar is therefore often referred to as a ‘scaffold modification’. The original pentose sugar may be replaced in its entirety by another moiety that similarly connects the base and the phosphate. It is therefore understood that while a pentose sugar is often a scaffold, a scaffold is not necessarily a pentose sugar. Examples of scaffold modifications that may be applied in the monomers of the AON as disclosed herein are disclosed in WO2020/154342, WO2020/154343, and WO2020/154344.

In one embodiment, the AON as disclosed herein may comprise one or more nucleotides carrying a 2′-MOE ribose modification. Also, in one embodiment, the AON comprises one or more nucleotides not carrying a 2′-MOE ribose modification, and wherein the 2′-MOE ribose modifications are at positions that do not prevent the enzyme with adenosine deaminase activity from deaminating the target adenosine. In another embodiment, the AON comprises 2′-OMe ribose modifications at the positions that do not comprise a 2′-MOE ribose modification, and/or wherein the oligonucleotide comprises deoxynucleotides at positions that do not comprise a 2′-MOE ribose modification. In one embodiment the AON comprises one or more nucleotides comprising a 2′ position comprising a 2′-MOE, 2′-OMe, 2′-OH, 2′-deoxy, TNA, 2′-fluoro (2′-F), 2′,2′-difluoro (diF) modification, 2′-fluoro-2′-C-methyl modification, or a 2′-4′-linkage (i.e., a bridged nucleic acid such as a locked nucleic acid (LNA or examples mentioned in e.g.

WO2018/007475)). In another embodiment, other nucleic acid monomer that are applied are arabinonucleic acids and 2′-deoxy-2′-fluoroarabinonucleic acid (FANA), for instance for improved affinity purposes. The 2′-4′ linkage can be selected from linkers known in the art, such as a methylene linker or constrained ethyl linker. A wide variety of 2′ modifications are known in the art. Further examples are disclosed in further detail in WO2016/097212, WO2017/220751, WO2018/041973, WO2018/134301, WO2019/219581, WO2019/158475, and WO2022/099159 for instance. In all cases, the modifications should be compatible with editing such that the AON fulfils its role as an oligonucleotide that can form a double stranded complex with the target RNA and by generating this double-stranded nucleic acid complex, recruit a deaminating enzyme, which can subsequently deaminate the target adenosine. Where a monomer comprises an unlocked nucleic acid (UNA) ribose modification, that monomer can have a 2′ position comprising the same modifications discussed above, such as a 2′-MOE, a 2′-OMe, a 2′-OH, a 2′-deoxy, a 2′-F, a 2′,2′-diF, a 2′-fluoro-2′-C-methyl, an arabinonucleic acid, a FANA, or a 2′-4′-linkage (i.e., a bridged nucleic acids such as a locked nucleic acid (LNA)).

A base, sometimes called a nucleobase, is generally adenine, cytosine, guanine, thymine or uracil, or a derivative thereof. A base, sometimes called a nucleobase, is defined as a moiety that can bond to another nucleobase through H-bonds, polarized bonds (such as through CF moieties) or aromatic electronic interactions. Cytosine, thymine, and uracil are pyrimidine bases, and are generally linked to the scaffold through their 1-nitrogen. Adenine and guanine are purine bases and are generally linked to the scaffold through their 9-nitrogen. The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar.

The nucleobases in an AON as disclosed herein can be adenine, cytosine, guanine, thymine, or uracil or any other moiety able to interact with another nucleobase through H-bonds, polarized bonds (such as CF) or aromatic electronic interactions. The nucleobases at any position in the nucleic acid strand can be a modified form of adenine, cytosine, guanine, or uracil, such as hypoxanthine (the nucleobase in inosine), pseudouracil, pseudocytosine, isouracil, N3-glycosylated uracil, 1-methylpseudouracil, orotic acid, agmatidine, lysidine, 2-thiouracil, 2-thiothymine, 5-substituted pyrimidine (e.g., 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine), 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, 8-oxo-adenine, 3-deazapurine (such as a 3-deaza-adenosine), pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp and its derivatives, Super A, Super T, Super G, amino-modified nucleobases or derivatives thereof; and degenerate or universal bases, like 2,6-difluorotoluene, or absent like abasic sites (e.g., 1-deoxyribose, 1,2-dideoxyribose, 1-deoxy-2-O-methylribose, azaribose).

In an embodiment, the nucleotide analog is an analog of a nucleic acid nucleotide. In an embodiment, the nucleotide analog is an analog of adenosine, guanosine, cytidine, thymidine, uridine, deoxyadenosine, deoxyguanosine, deoxycytidine, deoxythymidine or deoxyuridine. In an embodiment, the nucleotide analog is not guanosine or deoxyguanosine. In an embodiment, the nucleotide analog is not a nucleic acid nucleotide. In an embodiment, the nucleotide analog is not adenosine, guanosine, cytidine, thymidine, uridine, deoxyadenosine, deoxyguanosine, deoxycytidine, deoxythymidine, or deoxyuridine.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked scaffolds of an oligonucleotide is often called the ‘backbone’ of the oligonucleotide. Because phosphodiester bonds connect neighboring monomers together, they are often referred to as ‘backbone linkages’. It is understood that when a phosphate group is modified so that it is instead an analogous moiety such as a PS, such a moiety is still referred to as the backbone linkage of the monomer. This is referred to as a ‘backbone linkage modification’. In general terms, the backbone of an oligonucleotide comprises alternating scaffolds and backbone linkages.

As outlined in detail herein, AONs as disclosed herein comprise a linkage modification. Besides the linkage modification as depicted in the structure of formula (I), a linkage modification can be, but is not limited to, a modified version of the phosphodiester present in RNA, such as PS, chirally pure PS, (R)-PS, (S)-PS, MP, chirally pure MP, (R)-MP, (S)-MP, phosphoryl guanidine (such as PNdmi), chirally pure phosphoryl guanidine, (R)-phosphoryl guanidine, (S)-phosphoryl guanidine, phosphorodithioate (PS2), phosphonacetate (PACE), phosphonoacetamide (PACA), thiophosphonoacetate, thiophosphonoacetamide, methyl phosphorohioate, methyl thiophosphonate, PS prodrug, alkylated PS, H-phosphonate, ethyl phosphate, ethyl PS, boranophosphate, borano PS, methyl boranophosphate, methyl borano PS, methyl boranophosphonate, methyl boranophosphothioate, phosphate, phosphotriester, aminoalkylphosphotriester, and their derivatives. Another modification includes phosphoramidite, phosphoramidate, N3′→P5′ phosphoramidate, phosphorodiamidate, phosphorothiodiamidate, sulfamate, diethylenesulfoxide, amide, sulfonate, siloxane, sulfide, sulfone, formacetyl, alkenyl, methylenehydrazino, sulfonamide, triazole, oxalyl, carbamate, methyleneimino (MMI), and thioacetamide nucleic acid (TANA); and their derivatives. Various salts, mixed salts, deprotonated, protonated, tautomeric, and free acid forms are also included, as well as 3′→3′ and 2′→5′ linkages.

Apart from the one or more linkages according to formula (I), in one embodiment, an AON comprises a substitution of one of the non-bridging oxygens in the phosphodiester linkage. This modification slightly destabilizes base pairing but adds significant resistance to nuclease degradation. A preferred nucleotide analogue or equivalent comprises PS, phosphonoacetate, phosphorodithioate, phosphotriester, aminoalkylphosphotriester, H-phosphonate, methyl and other alkyl phosphonate including 3′-alkylene phosphonate, 5-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. Particularly preferred are internucleoside linkages that are modified to contain a PS.

Many of these non-naturally occurring modifications of the linkage, such as PS are chiral, which means that there are Rp and Sp configurations, known to the person skilled in the art. In one embodiment, the chirality of the PS linkages is controlled, which means that each of the linkages is either in the Rp or in the Sp configuration, whichever is preferred. The choice of an Rp or Sp configuration at a specified linkage position may depend on the target sequence and the efficiency of binding and induction of providing RNA editing. However, if such is not specifically desired, a composition may comprise AONs as active compounds with both Rp and Sp configurations at a certain specified linkage position. Mixtures of such AONs are also feasible, wherein certain positions preferably have either one of the configurations, while for other positions such does not matter.

Again, in all cases, the modifications should be compatible with editing such that the AON fulfils its role as an oligonucleotide that can, after binding to its target sequence, recruit an adenosine deaminase enzyme because of the double-stranded nucleic acid entity that arises. In all aspects of the disclosure, the enzyme with adenosine deaminase activity is preferably ADAR1, ADAR2, or ADAT. In a highly preferred embodiment, the AON is an oligonucleotide that targets a pre-mRNA or an mRNA, wherein the target nucleotide is an adenosine in the target RNA, wherein the adenosine is deaminated to an inosine, which is being read as a guanosine by the translation machinery. Disclosed is also a pharmaceutical composition comprising the AON as characterized herein, and a pharmaceutically acceptable carrier.

Other chemical modifications of the AON as disclosed herein include the substitution of one or more than one of any of the hydrogen atoms with deuterium or tritium, examples of which can be found in e.g., WO2014/022566 or WO2015/011694.

AONs as disclosed herein preferably do not include a 5′-terminal 06-benzylguanosine or a 5′-terminal amino modification and preferably are not covalently linked to a SNAP-tag domain (an engineered 06-alkylguanosine-DNA-alkyl transferase). AONs as disclosed herein preferably do not comprise a boxB RNA hairpin sequence. In one embodiment, an AON as disclosed herein comprises 0, 1, 2 or 3 wobble base pairs with the target sequence, and/or 0, 1, 2, 3, 4, 5, 6, 7, or 8 mismatching base pairs with the target RNA sequence. No mismatch exists when the orphan nucleotide is uridine. One alternative for uridine is positioning an iso-uridine opposite the target adenosine, which likely does not pair like G pairs with U. Preferably, the target adenosine in the target sequence forms a mismatch base pair with the nucleoside in the AON that is directly opposite the target adenosine.

An AON as disclosed herein can utilise endogenous cellular pathways and naturally available ADAR enzymes to specifically edit a target adenosine in the target RNA sequence. An AON as disclosed herein is capable of recruiting ADAR and complex with it and then facilitates the deamination of a (single) specific target adenosine nucleotide in a target RNA sequence to which it is bound. Ideally, only one adenosine is deaminated. An AON as disclosed herein, when complexed to ADAR, preferably brings about the deamination of a single target adenosine.

Analysis of natural targets of ADAR enzymes has indicated that these generally include mismatches between the two strands that form the RNA helix edited by ADAR1 or 2. It has been suggested that these mismatches enhance the specificity of the editing reaction (Stefl et al. 2006. Structure 14(2):345-355; Tian et al. 2011. Nucleic Acids Res 39(13):5669-5681). Characterization of optimal patterns of paired/mismatched nucleotides between the AONs and the target RNA also appears important to the development of efficient ADAR-based AON therapy.

As outlined above, an AON as disclosed herein makes use of specific nucleotide modifications at predefined spots to ensure stability as well as proper ADAR binding and activity. These changes may vary and may include modifications in the backbone of the AON, in the sugar moiety of the nucleotides as well as in the nucleobases or the phosphodiester linkages, as outlined in detail herein. They may also be variably distributed throughout the sequence of the AON. Specific modifications may be needed to support interactions of different amino acid residues within the RNA-binding domains of ADAR enzymes, as well as those in the deaminase domain. For example, PS linkages between nucleotides or 2′-OMe or 2′-MOE modifications may be tolerated in some parts of the AON, while in other parts they should be avoided so as not to disrupt crucial interactions of the enzyme with the phosphate and 2′-OH groups. Specific nucleotide modifications may also be necessary to enhance the editing activity on substrate RNAs where the target sequence is not optimal for ADAR editing. Previous work has established that certain sequence contexts are more amenable to editing. For example, a target sequence 5′-UAG-3′ (with the target A in the middle) contains the most preferred nearest-neighbor nucleotides for ADAR2, whereas a 5′-CAA-3′ target sequence is disfavored (Schneider et al. 2014. Nucleic Acids Res 42(10):e87). The structural analysis of ADAR2 deaminase domain hints at the possibility of enhancing editing by careful selection of the nucleotides that are opposite to the target trinucleotide. For example, the 5′-CAA-3′ target sequence, paired to a 3′-GCU-5′ sequence on the opposing strand (with the A-C mismatch formed in the middle), is disfavored because the guanosine base sterically clashes with an amino acid side chain of ADAR2. The guanosine opposite the C in such circumstances is preferably replaced by an inosine (hence, at the −1 position within the AON), more preferably a deoxyinosine.

Mutagenesis studies of human ADAR2 revealed that a single mutation at residue 488 from glutamate to glutamine (E488Q), gave an increase in the rate constant of deamination by 60-fold when compared to the wild-type enzyme (Kuttan and Bass. Proc Natl Acad Sci USA 2012. 109(48):3295-3304). During the deamination reaction, ADAR flips the edited base out of its RNA duplex, and into the enzyme active site (Matthews et al. Nat Struct Mol Biol. 2016. 23(5):426-433). When ADAR2 edits adenosines in the preferred context (an A:C mismatch) the nucleotide opposite the target adenosine is often referred to as the ‘orphan cytidine’, as indicated above. The crystal structure of ADAR2 E488Q bound to double stranded RNA (dsRNA) revealed that the glutamine (Gln; Q) side chain at position 488 can donate an H-bond to the N3 position of the orphan cytidine, which leads to the increased catalytic rate of ADAR2 E488Q. In the wild-type enzyme, wherein a glutamate (or glutamic acid; Glu; E) is present at position 488 instead of a glutamine (Gln) the amide group of the glutamine is absent and is instead a carboxylic acid. To obtain the same contact of the orphan cytidine with the E488Q mutant would then, for the wild-type situation, require protonation for this contact to occur. To make use of endogenously expressed ADAR2 to correct disease relevant mutations, it is essential to maximize the editing efficiency of the wild type ADAR2 enzyme present in the cell. WO2020/252376 discloses the use of AONs with modified RNA bases, especially at the position of the orphan cytidine to mimic the hydrogen-bonding pattern observed by the E488Q ADAR2 mutant. By replacing the nucleotide opposite the target adenosine in the AON with cytidine analogs that serve as H-bond donors at N3, it was envisioned that it would be possible to stabilize the same contact that is believed to provide the increase in catalytic rate for the mutant enzyme. Two cytidine analogs were of particular interest: pseudoisocytidine (also referred to as ‘piC’; Lu et al. J Org Chem 2009. 74(21):8021-8030; Burchenal et al. (1976) Cancer Res 36:1520-1523) and Benner's base Z (also referred to as ‘dZ’; Yang et al. Nucl Acid Res 2006. 34(21):6095-6101) that were initially selected because they offer hydrogen-bond donation at N3 with minimal perturbation to the shape of the nucleobase. Benner's base is also referred to with its chemical name 6-amino-5-nitro-3-yl-2(1H)-pyridone. The presence of the cytidine analog in the AON may exist in addition to modifications to the ribose 2′ group. The ribose 2′ groups in the AON can be independently selected from 2′-H (i.e., DNA), 2′-OH (i.e., RNA), 2′-OMe, 2′-MOE, 2′-F, or 2′-4′-linked (i.e., a bridged nucleic acid such as a locked nucleic acid (LNA)), or other 2′ substitutions. The 2′-4′ linkage can be selected from linkers known in the art, such as a methylene linker or constrained ethyl linker.

In one embodiment, a nucleotide analogue or equivalent within the AON comprises one or more base modifications or substitutions. Modified bases comprise synthetic and natural bases such as inosine, xanthine, hypoxanthine and other-aza, deaza, -hydroxy, -halo, -thio, thiol, -alkyl, -alkenyl, -alkynyl, thioalkyl derivatives of pyrimidine and purine bases that are or will be known in the art. Purine nucleobases and/or pyrimidine nucleobases may be modified to alter their properties, for example by amination or deamination of the heterocyclic rings. The exact chemistries and formats may vary from oligonucleotide construct to oligonucleotide construct and from application to application, and may be worked out in accordance with the wishes and preferences of those of skill in the art.

An AON as disclosed herein is normally longer than 10 nucleotides, preferably more than 11, 12, 13, 14, 15, 16, still more preferably more than 17 nucleotides. In one aspect the AON as disclosed herein is longer than 20 nucleotides. The AON as disclosed herein is preferably shorter than 100 nucleotides, still more preferably shorter than 60 nucleotides, still more preferably shorter than 50 nucleotides. In a preferred aspect, the AON as disclosed herein comprises 18 to 70 nucleotides, more preferably comprises 18 to 60 nucleotides, and even more preferably comprises 18 to 50 nucleotides. Hence, in a particularly preferred aspect, the AON as disclosed herein comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides. In one embodiment, the AON is 27, 28, 29, or 30 nucleotides in length.

In one aspect, at either end or both termini of an AON as disclosed herein, inverted deoxyT or dideoxyT nucleotides are incorporated.

After RNA editing has occurred in a cell, the modified RNA can become diluted over time, for example due to cell division, limited half-life of the edited RNAs, etc. Thus, in practical therapeutic terms a method as disclosed herein may involve repeated delivery of an AON until enough target RNAs have been modified to provide a tangible benefit to the patient and/or to maintain the benefits over time.

### EXAMPLES

### Example 1. Synthesis of Oligonucleotides Carrying PNms at a Variety of Positions

The synthesis of the PNms compound for incorporation into oligonucleotides was performed as follows, using standard protocols known to the person skilled in the art. 20 mL acetone (anhydrous) was added to 1.95 g NaN₃ (1.50 eq, 30.0 mmol) in a 100 mL round bottom flask under inert atmosphere (N₂). The suspension was cooled to 0° C. 1.55 mL methanesulfonyl chloride (1.00 eq, 20.0 mmol, 2.29 g) was added min to the stirring suspension. The reaction mixture was allowed to warm up to RT. Then the reaction mixture was filtrated, and the salt residue washed with anhydrous acetone (transfer and wash volume together −5 mL). Then the solvent was removed under reduced pressure at 30° C. to give pure mesyl azide as clear, colour less liquid in near quantitative yields (96-97%). The mesyl azide building block was incorporated into the oligonucleotide using general methods known to the person skilled in the art.

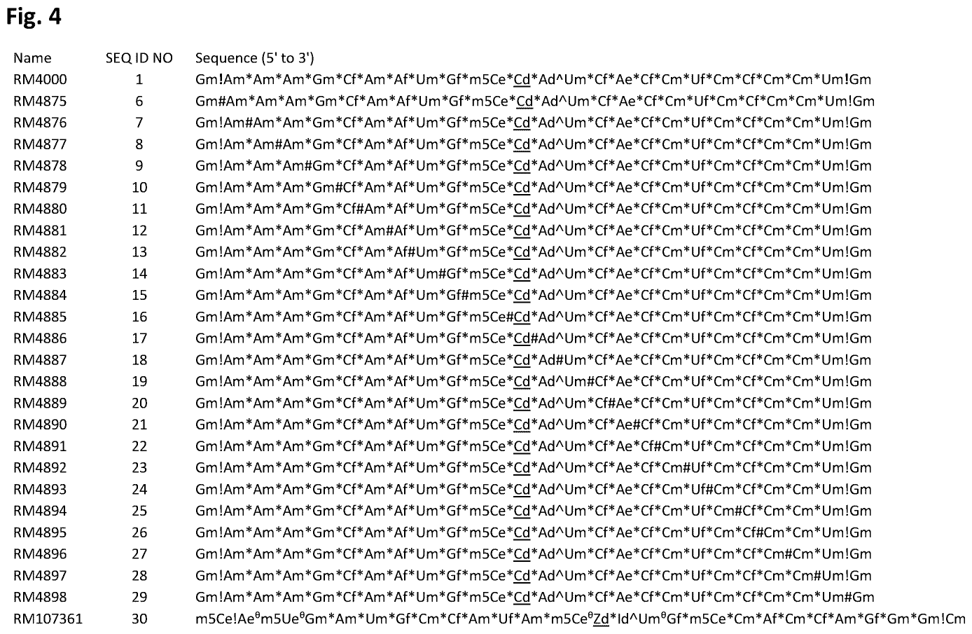

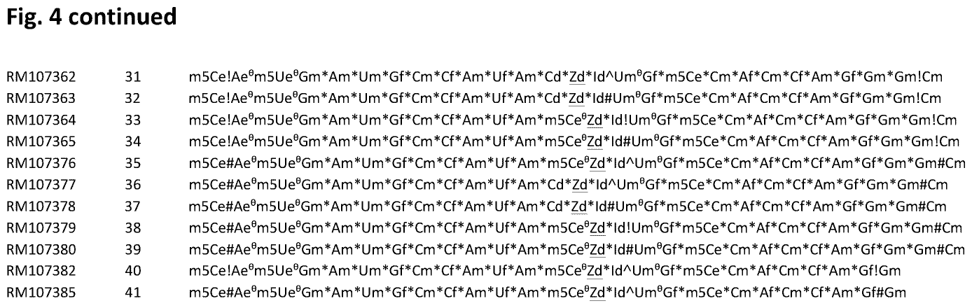

An initial set of 24 human Actin B transcript-targeting antisense oligonucleotides (100 μM stocks) were designed and generated (RM4875 to RM4898; SEQ ID NO:6 to 29, respectively), in which the potential of having a PNms linkage in each linkage position of a reference oligonucleotide (RM4000) was investigated. The sequences and chemical modifications of AONs RM4000, and RM4875 to RM4898 are provided in FIG. 4, wherein the cytidine opposite the target adenosine in the human Actin B target transcript is the orphan nucleotide and is underlined; wherein the internucleoside linkage numbering is such that linkage number 0 is the linkage 5′ from the orphan nucleotide, and the linkage positions in the oligonucleotide are positively (+) incremented towards the 5′ end and negatively (−) incremented towards the 3′ end. This counting holds true throughout the present disclosure. The other modifications are as described in the legend to FIG. 4.

The only difference between RM4000 and each of the oligonucleotides RM4878 to RM4898 is in each case one specific linkage difference using PNms (generally referred to in a sequence with a hashtag symbol “#”). Since there are 24 internucleoside linkages in RM4000, 24 differences from 5′ to 3′ were generated in comparison to RM4000. Hence, RM4875 has a PNms linkage at linkage position +10, RM4876 has a PNms linkage at linkage position +9 and so further, while RM4885 has a PNms linkage at linkage position 0 and RM4898 has a PNms linkage at linkage position −13.

### Example 2. Editing of a Target Adenosine in a Human Actin B Target RNA Molecule in Cells

Human retinoblastoma cell line Weri-rb1 cells carrying the wildtype UTR editing region of Actin B were obtained from DSMZ (ACC-90). Cells were cultured in RPM11640 medium (Gibco) +10% FBS (Biowest)+1% pen/strep (Sigma) and incubated at 37° C./5% CO₂. For the editing assays, 1×10⁵ cells/well were plated in a 24-well plate in the same culture medium. Cells were then treated with 5 μM of AON on day 0. Non-treated Weri-rb1 cells were taken as a negative control. After incubating the cells for a subsequent period of 5 days, cells were collected. The culture medium was removed, and cells washed with cold PBS (Gibco) and lysed with Trizol (Ambion). Additionally, bottoms of the wells were scraped to ensure sufficient cell lysing. Cell lysates were stored at −25° C. until RNA isolation.

RNA was isolated from the cells using the Direct-zol RNA Microprep kit (Zymo Research) kit according to the manufacturer's instructions. The RNA yield was determined using spectrophotometric analysis (NanoDrop) and stored at −80° C.

Maxima Reverse Transcriptase (RT, ThermoFisher) was used to generate cDNA. Typically, 400 nanogram total RNA was used in a reaction mixture containing 2 μL dNTP mix (10 mM each), 0.5 μL Oligo(dT), 0.5 μL random hexamer (all ThermoFisher) supplemented with DNase- and RNase-free water to a total volume of 15.5 μL. Samples were loaded in a T100 thermocycler (Bio-Rad) for a pre-heating step of 5 min at 65° C. to detach AONs that might be still bound to the target. Then 4 μL of 5x RT buffer and 0.5 μL of the Maxima RT enzyme were added to the reaction mix. The samples were again loaded in a T100 thermocycler (Bio-Rad) and initially incubated at 10 min at 25° C., followed by a cDNA reaction temperature of 50° C. (30 min) and a termination step of 5 min at 85° C. Samples were cooled down to 4° C. prior storing at −20° C.

To determine the editing efficiency, cDNA samples were used in multiplex dPCR (Qiagen) assays. The primer and probe sequences were:

(human ActB forward primer; SEQ ID NO: 2)
5′-AGTCCTCTCCCAAGTCCACA-3′
(human ActB reverse primer; SEQ ID NO: 3)
5′-GGCACGAAGGCTCATCATTC-3′
(probe target G; SEQ ID NO: 4)
FAM-AGGTGA+T+G+GCATTGCTTTCGT-IABKFQ
(probe target A; SEQ ID NO: 5)
HEX-AG+GTGA+T+A+GCATTGCTTTCGTGT-IABKFQ

Herein, in respect of the probes, the “+” symbol indicates a Locked Nucleic Acid (LNA) at the 3′ side of the symbol. In total, 1.2 μL of the cDNA mix was used in a dPCR mixture containing 3 μL 4×dPCR QIAcuity Mastermix for probes (Qiagen), 0.6 μL primers and 0.3 μL probes (10 μM stock concentration each), supplemented with 6 μL DNase- and RNase-free water in a total volume of 12 μL. The resulting mixture was mixed thoroughly and transferred to a well of a QIAcuity 96-wells 8.5K Nanoplate (Qiagen) and loaded in a QIAcuity dPCR machine using general cycling conditions. Data was analyzed using the QIAcuity Software Suit (Qiagen). Percentage of A-to-I editing was determined by dividing the number of G-containing molecules by the total (G- plus A-containing species) multiplied by 100.

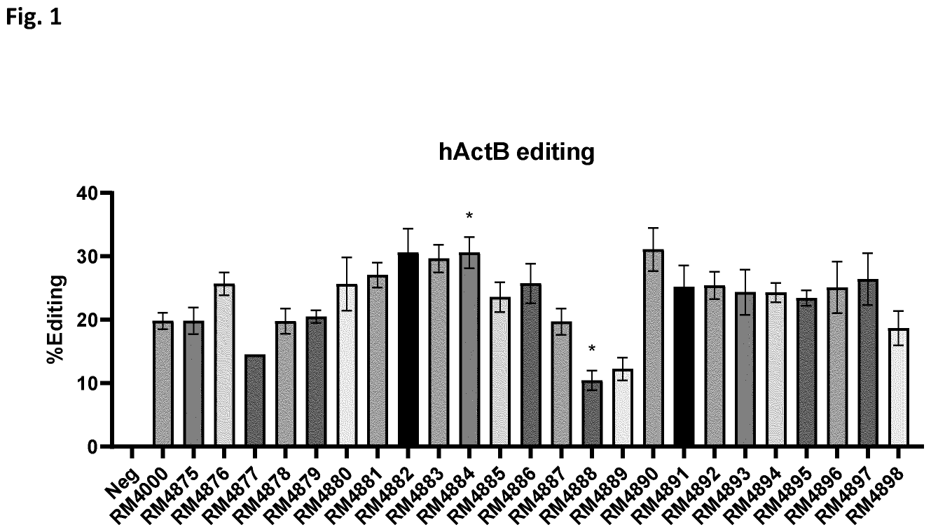

The average A-to-I editing percentage results are shown in FIG. 1. This indicates that the PNms linkage is tolerated well during editing of the target adenosine in the target human ActB target transcript. The asterisks indicate a significant difference in comparison to the RM4000 control. The results indicate that PNms can replace PNdmi at both terminal linkages (comparing RM4875 and RM4898 to RM4000). The results also indicate that PNms can be used (instead of PS) in almost all linkage positions in the AON without affecting the editing percentage. Notably, when a PNms linkage was used at linkage positions −3 and −4 (RM4888 and RM4889, respectively), thereby replacing PS, a decrease in editing efficiency was observed. This suggests that a PNms linkage at either one of those positions interferes with ADAR binding and the deamination process. Importantly, it was investigated whether PNms could be used instead of MP at linkage position −2, predominantly because MP is known to be a difficult linkage modification for manufacturing purposes, even though it has beneficial editing properties over AONs that comprise a PS or PO at linkage position −2. The representative AON in which this MP is replaced (or wherein a PNms is used instead of the MP linkage) is RM4887. In view of the results with using PNms on linkage positions −3 and −4, see above, it was surprising to find that a PNms linkage at linkage position −2 was well-tolerated and provided editing efficiencies that were comparable to RM4000. This shows that it is now possible to replace MP with PNms and still obtain sufficient editing.

### Example 3. Stability Study of RM4000 and RM4887 in PBS and aCSF

It was then questioned whether using mesyl phosphoramidate (PNms) at linkage position −2 instead of methyl phosphonate (MP) would influence the stability of the AON. For this, the long-term stability was tested for the reference RM4000 AON in comparison to RM4887 (see FIG. 4) that only differ in the linkage at linkage position −2, using a variety of temperatures and time. A stability study was designed with the goal of assessing the difference in chemical stability between the MP and PNms linker. The MP linker has been shown to get cleaved over time at elevated temperatures (other studies, data not shown). This rate of degradation appeared even higher in artificial cerebrospinal fluid (aCSF) than in PBS. Hence, the stability was studied using aCSF and PBS at different temperatures, over time.

Both RM4000 and RM4887 were dissolved in PBS and in aCSF and formulated at 0.3 mg/mL. The study consisted of two time points: 4 and 8 weeks, and three storage conditions: 5° C., 25° C. and 40° C. The purity of the material at t=0 was determined in aCSF as the solvent was not expected to impact the HPLC analysis for the purity determination. HPLC analysis was performed using Ion-Pair Reversed Phase (IPRP) UPLC combined with UV detection and mass spectrometry (MS) analysis. IPRP is based on the electrostatic interaction between an oligonucleotide and an ion pairing reagent that is added to the mobile phase, such as tri- or tetra-alkylamine salts, and the hydrophobic interactions between the ion pairing reagent and the non-polar stationary phase. For this method, triethylamine (TEA) and hexafluoro-isopropanol (HFIP) was used as ion-pairing reagent and methanol as organic modifier. UV detection was used at a wavelength of 260 nanometers and MS detection for the identification. The purity of RM4000 at t=0 was 95.7%. The purity of RM4887 at t=0 was 97.6%. The degree of purity over time under the different conditions was determined with the HPLC method described above and then graphically displayed with the percentage purity over time. The results are shown in FIGS. 2A and 2B for the purity (stability) of each AON in PBS and aCSF, respectively. Very surprisingly, it was found that RM4887, carrying the PNms linkage at linkage position −2 remained stable during the course of 8 weeks, even at 40° C., in PBS as well as in aCSF, in contrast to RM4000 that degraded significantly to 60-70% in the course of 8 weeks. In PBS, RM4000 remained relatively stable over time at all three temperatures, but in aCSF it degraded also at 25° C. in 8 weeks. This shows that the PNms linkage at linkage position −2, thereby replacing the MP linkage that is often used at this position, appears to contribute significantly to the stability of the AON, both in PBS and in aCSF at elevated temperatures for a longer duration of time. It is held that this is not only true for the linkage position −2, but should be valid also at other positions where an MP linkage can be present. It is held that the replacement of the MP at any position in an AON by a PNms linkage will increase the stability of the AON, and it is held that concomitantly this will contribute to the efficiency and longer lasting RNA editing effects, in vitro, ex vivo, and in vivo.

### Example 4. Editing of a Target Adenosine in the Q68R Target RNA Molecule in PHH

Next, it was questioned whether a PNms linkage could also be used as an alternative for other linkages than MP and PS, as discussed above. To test this, a set of AONs was designed that target an adenosine in the human SLC10A1 transcript, encoding the Na⁺/Taurocholate Co-transporting Polypeptide (NTCP; see GB2304438.1 and GB2318087.0, unpublished). The deamination of the target adenosine in this target transcript changes a CAG codon to a CIG codon, which is subsequently read by the translation machinery as CGG, which then yields a change from glutamine (Q) to arginine (R) at position 68 in the human NTCP protein. The twelve AONs that were designed to determine the effect of having PO, PS, MP, PNdmi or PNms linkages in the AONs are RM107361 to RM107365, RM107376 to 107380, RM107382, and RM107385, and are shown in detail in FIG. 4.

To determine the editing efficiency of these AONs, primary human hepatocytes (PHH's, Primacyte) that express the wild-type human SLC10A1 transcript, were transfected with 100 nM of each AON using Lipofectamine 2000 (Thermo Fisher Scientific) according to the manufacturer's specifications. Approximately 2×10⁵ cells per well were incubated for 72 hr at 37° C. and 5% CO₂. Then, total RNA was isolated using the Reliaprep total RNA isolation system (Promega; Z6012). cDNA was subsequently synthesized using the Maxima Reverse Transcriptase kit using a mixture of random hexamers. The percentage of ADAR-mediated A-to-I conversion was determined by quantitative digital PCR (dPCR) assays designed for the NTCP Q68R target site, using the primers and probes provided in Table I.

The percentage editing was calculated by dividing the guanidine (G)-containing cDNA species by the total number of target copies, multiplied by 100. A control dPCR was performed upstream or downstream of the transcript for standardization purposes. The entire experiment was performed in triplicate and editing percentages were averaged.

TABLE I
Primers and probes for quantitative dPCR assays. The SEQ ID NO of each primer or probe is
given between brackets following the respective sequence in the middle column. The + indicates a
Locked Nucleic Acid (LNA) on the 3′ side of the symbol.

| Name | Sequence 5′-3′ (SEQ ID NO) | Item |
|---|---|---|
| hSLC10A1_e01_fw1 | GTTCAGCAAGATCAAGGCTCAC (42) | Primer |
| hSLC10A1_e01_edit(G)_FAM | CTGG+TGGCA+C+G+GTATGGCATCAT (43) | Probe |
| hSLC10A1_e01_orig(A)_HEX | CTGG+TGGCA+C+A+GTATGGCA+TCAT (44) | Probe |
| hSLC10A1_e01_TEX | CTGG+TGGCA+C+A+GTATGGCA+TCAT (45) | Probe |
| hSLC10A1_e02_rv1 | TGGAGCAGGTGGTCATCACAA (46) | Primer |

The results of the editing assay in transfected PHH's are provided in FIG. 3. It should be noted that RM107361 to RM107365, and RM107382 all carry PNdmi linkages on the 5′ and 3′ terminal linkage positions and gave editing percentages ranging from around 6-11%. However, quite remarkably, when instead of these PNdmi linkages, PNms linkages were introduced at the 5′ and 3′ terminal positions the editing percentages increased to 13-18%. Even more surprising, but in line with the stability results and the editing results from the previous examples, when also the MP linkage at linkage position −2 was replaced by PNms (compare RM107378 with RM107362, wherein RM107378 does not contain PNdmi and MP linkages), the editing efficiency was highest. This clearly shows the beneficial properties of applying PNms linkages in oligonucleotides that provide RNA editing through an endogenous ADAR enzyme, naturally present in cells, because it cannot only contribute to an increased stability (in comparison to oligonucleotides carrying an MP linkage instead), but also in increased editing efficiency, which effects may in fact go hand in hand.

1. An antisense oligonucleotide (AON) that is able to mediate deamination of a target adenosine in a cell by recruitment of an endogenous ADAR enzyme after the AON has formed a double-stranded complex with a region of a target RNA nucleic acid molecule in the cell, wherein the region comprises the target adenosine, wherein the ADAR enzyme can deaminate the target adenosine into an inosine, and wherein the AON comprises one or more linkage modifications with a structure according to formula (I): 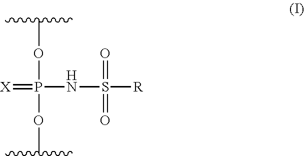
wherein: X=O or S; and R=an aryl, a substituted aryl, a heterocycle, a substituted heterocycle, an aromatic heterocycle, a substituted aromatic heterocycle, a C₁-C₆ alkoxy, a substituted C₁-C₆ alkoxy, a C₁-C₂₀ alkyl, a substituted C₁-C₂₀ alkyl, a C₁—C alkenyl, a C₁-C₆ substituted alkenyl, a C₁-C₆ alkynyl, a substituted C₁-C₆ alkynyl, or a conjugate group.

2. An AON according to claim 1, wherein at least one of the linkage modifications comprises a structure according to formula (I), wherein X=O and R=CH₃.

3. An AON according to claim 1 or 2, wherein the nucleotide that is directly opposite the target adenosine is the orphan nucleotide, wherein the internucleoside linkage numbering is such that linkage number 0 is the linkage 5′ from the orphan nucleotide, the linkage positions in the oligonucleotide are positively (+) incremented towards the 5′ end and negatively (−) incremented towards the 3′ end, and wherein the linkage modification according to formula (I) is present at:
linkage position −2;
the linkage position connecting the 5′ terminal two nucleosides in the AON; and/or
the linkage position connecting the 3′ terminal nucleosides in the AON.

4. An AON according to any one of claims 1 to 3, comprising at least one additional non-naturally occurring chemical modification in the ribose, linkage, or base moiety.

5. An AON according to claim 4, wherein the additional modification is an internucleoside linkage modification selected from the group consisting of a: phosphorothioate (PS), phosphonoacetate, phosphorodithioate, methylphosphonate (MP), phosphoryl guanidine, phosphoramidate, sulfonylphosphoramidate, and PNdmi internucleotide linkage.

6. An AON according to claims 4 or 5, wherein the one or more additional modifications in the ribose moiety is a mono- or di-substitution at the 2′, 3′ and/or 5′ position of the ribose, each independently selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy.

7. A pharmaceutical composition comprising an AON according to any one of claims 1 to 6, and a pharmaceutically acceptable carrier, solvent, or diluent.

8. A method for the deamination of a target adenosine, present in a target RNA nucleic acid molecule in a mammalian cell, the method comprising the steps of:
(i) providing the cell with an AON according to any one of claims 1 to 6, or a pharmaceutical composition according to claim 7;
(ii) allowing annealing of the AON to the target RNA nucleic acid molecule;
(iii) allowing a deaminating enzyme, preferably naturally present in the cell, to deaminate the target adenosine in the target RNA nucleic acid molecule; and
(iv) optionally identifying the presence of the deaminated adenosine in the target RNA nucleic acid molecule.

9. The method of claim 8, wherein step (iv) comprises:
a. sequencing a region of the target RNA molecule, wherein the region comprises the deaminated target nucleotide;
b. assessing the presence of a functional, elongated, full length and/or wild type protein when the target nucleotide is an adenosine located in a UGA or UAG stop codon;
c. assessing, when the target RNA molecule is pre-mRNA, whether splicing of the pre-mRNA was altered by the deamination; or
d. using a functional read-out, wherein the target RNA molecule after the deamination encodes a functional, full length, elongated and/or wild type protein.

10. A method according to claim 8 or 9, further comprising the step of administering to the subject a therapeutically effective dose of a saponin, preferably the triterpene glycoside AG1856.

11. An AON that is able to mediate deamination of a target adenosine in a cell by recruitment of an endogenous ADAR enzyme after the AON has formed a double-stranded complex with a region of a target RNA nucleic acid molecule in the cell, wherein the region comprises the target adenosine, wherein the ADAR enzyme can deaminate the target adenosine into an inosine, and wherein the AON comprises a terminal moiety at one or both termini with a structure according to formula (II): 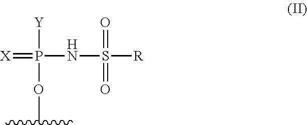
wherein: X=O or S; Y=O⁻ or S⁻; and R=an aryl, a substituted aryl, a heterocycle, a substituted heterocycle, an aromatic heterocycle, a substituted aromatic heterocycle, a C₁-C₆ alkoxy, a substituted C₁-C₆ alkoxy, a C₁-C₂₀ alkyl, a substituted C₁-C₂₀ alkyl, a C₁-C₆ alkenyl, a C₁-C₆ substituted alkenyl, a C₁-C₆ alkynyl, a substituted C₁-C₆ alkynyl, or a conjugate group.

12. An AON according to claim 11, wherein one or both terminal moieties comprise a structure according to formula (II), wherein X=O and R=CH₃.
